(19) United States
(12) Patent Application Publication
(10) Pub. No.: US 2024/0002463 A1
(43) Pub. Date: Jan. 4, 2024

(54) CHEMOGENETIC RECEPTORS AND METHODS OF MAKING AND USING

(71) Applicant: Howard Hughes Medical Institute, Chevy Chase, MD (US)

(72) Inventors: Scott Sternson, Chevy Chase, MD (US); Christopher Magnus, Crozet, VA (US)

(21) Appl. No.: 18/140,403

(22) Filed: Apr. 27, 2023

Related U.S. Application Data
(60) Provisional application No. 63/335,249, filed on Apr. 27, 2022.

(51) Int. Cl. C07K 14/705; C12N 5/0793; A61P 39/02

(52) U.S. Cl. CPC C07K 14/705; A61P 39/02; C12N 5/0619

(57) ABSTRACT

This disclosure describes a number of chemogenetic receptors that bind an ingested substance that reinforces its own ingestion or administration (e.g., an addictive drug) and, upon binding of the molecule, modulate the function of a cell.

### CROSS REFERENCE TO RELATED APPLICATIONS

This application claims the benefit of priority to U.S. Application No. 63/335,249 filed Apr. 27, 2022. This document is incorporated by reference herein.

### TECHNICAL FIELD

This disclosure generally relates to chemogenetic receptors.

### BACKGROUND

Drug addiction is a major public health problem in the United States. In 2020, drug overdose deaths rose by 29%. Cocaine is a powerfully addictive psychostimulant and a Schedule II drug. In 2014, there were approximately 1.5 million current cocaine users aged 12 or older (˜0.6% of the U.S. population) with 1.4% of young adults (18-25 yrs) reporting current cocaine use. As of 2017, nearly 1 million people in the U.S. were diagnosed with methamphetamine used disorder. In addition, opioids comprise another powerfully addictive class of drugs. Most opioids have therapeutic use and are Schedule II, though heroin and certain non-therapeutic opioids are Schedule I. In 2019, 1.6 million people reported an opioid use disorder, and over 10 million people reported misusing prescription opioids in the past year. Moreover, 460,000 Americans die each year due to nicotine addiction in the form of cigarette smoking.

Treatment of substance use disorder (SUD) is complicated by the fact that the neural circuits are associated with natural rewards. New chemogenetic receptors are described herein that are activated specifically by addictive drugs. The resulting chemogenetic receptors allow investigation of SUD-related neurobiology and behavior by selective negative-feedback neuromodulation, advance understanding of neural control over drug seeking, and provide a potential avenue for new gene therapy approaches for selectively treating SUDs.

### SUMMARY

Traditionally, discovery of small molecule modulators for endogenous signaling pathways starts with a receptor and identifies small molecule modulators. Most applications of small molecules in vivo influence many cell populations, complicating mechanistic interpretation. Chemogenetics inverts this framework such that a molecule (e.g., an addictive drug) is identified and then a molecule-specific receptor is developed that can be delivered by viral methods to localized neural circuit nodes (e.g., specific neuron populations).

Modulation of the neural processes underlying drug addiction by traditional pharmacology or chemogenetics involves open-loop neuronal perturbations that precede ingestion of a drug and are not directly influenced by drug-taking. Negative-feedback chemogenetics fills this gap by developing closed-loop perturbation tools to modify neural circuits in concert with the endogenous effects of addictive drugs. There has been progress with related approaches using electrical or optogenetic stimulation, which can be controlled on millisecond timescales. Despite high temporal precision of these methods, however, they lack the capability to precisely track the pharmacokinetics of drug exposure in different brain regions, which is an inherent component of negative-feedback chemogenetics.

Drug-controlled chemogenetic receptors are potential gene therapies for drug addiction. Current therapeutic approaches for treating cocaine addiction include drug replacement therapy with amphetamine or buproprion, cocaine vaccine, and gene therapy for an engineered butyrylesterase enzyme for cocaine hydrolysis targeted to liver cells. Drug-controlled chemogenetic gene therapies are a novel approach that have the potential to modify the neural circuits that respond to drug seeking and attain long-term drug avoidance.

In one aspect, an engineered human chemogenetic cell-surface receptor is provided. Such a receptor typically includes a ligand binding domain (LBD) and an activation domain, wherein the LBD has been engineered to bind a ligand associated with a substance (e.g., a controlled substance such as an addictive drug or a nutrient substance such as sugar or fatty acids) whose ingestion results in reinforcing behavior.

Representative controlled substances include, without limitation, cocaine or cocaine metabolites, methylphenidate (Ritalin), amphetamines (e.g., amphetamine, MDMA, and methamphetamine), cathinones (e.g., bupropion, MDPV, mephedrone, and methylone), and opioids (e.g., morphine, oxycodone, dihydrocodeine, heroin, methadone, and fentanyl).

In some embodiments, the cell-surface receptor is a ligand gated ion channel (LGIC) or a G-protein coupled receptor (GPCR). In some embodiments, the LBD is a mutated alpha-7-5HT3 LBD. In some embodiments, the LBD is a mutated alpha 7-GlyR LBD.

In some embodiments, the engineered human chemogenetic cell-surface receptor has the sequence shown in SEQ ID NO:1 or SEQ ID NO:2 having one or more of the substitutions shown in Table 2.

In another aspect, cells comprising the engineered human chemogenetic cell-surface receptor described herein are provided. In some embodiments, the cell is a neuron. In some embodiments, the cell is in culture. In some embodiments, the cell is in vivo.

In still another aspect, methods of treating a disorder associated with the use of a substance (e.g., a controlled substance such as an addictive drug or a nutrient substance such as sugar or fatty acids) are provided. Such methods typically include delivering an engineered human chemogenetic cell-surface receptor as described herein to an individual, wherein, in the presence of the substance, the engineered human chemogenetic cell-surface receptor reduces a reward response for the substance or increases an aversion response for the substance.

In some embodiments, the engineered human chemogenetic cell-surface receptor is delivered in the form of a nucleic acid encoding the engineered human chemogenetic cell-surface receptor.

Representative controlled substances include, without limitation, cocaine or cocaine metabolites, methylphenidate (Ritalin), amphetamine (e.g., amphetamine, MDMA, and methamphetamine), cathinone (e.g., bupropion, MDPV, mephedrone, and methylone), and opioid (e.g., morphine, oxycodone, dihydrocodeine, heroin, methadone, and fentanyl).

In yet another aspect, engineered ligand gated ion channels (LGICs) having a plurality of engineered LGIC subunits are provided, wherein each of the plurality of engineered LGIC subunits includes: (a) a ligand binding domain (LBD), wherein the LBD binds a ligand associated with a substance use disorder; and (b) an ion pore domain (IPD), wherein the IPD is selected from a serotonin 3 receptor (5HT3) IPD or a glycine receptor (GlyR) IPD; wherein the presence of the ligand activates or deactivates the engineered LGIC.

In still another aspect, engineered ligand gated ion channels (LGICs) are provided that include at least one engineered LGIC subunit. As described herein, the engineered LGIC subunits includes (a) an alpha7 nicotinic acetylcholine receptor (alpha-7-nAChR) ligand binding domain (LBD) mutated to bind a ligand associated with a substance use disorder; and (b) an ion pore domain (IPD), wherein the IPD is selected from the group consisting of a serotonin 3 receptor (5HT3) IPD or a glycine receptor (GlyR) IPD. Generally, the presence of the ligand activates or deactivates the engineered LGIC.

In some embodiments, the mutated LBD is mutated at Trp77, Gln79, Tyr115, Leu131, Gln139, Leu141, Val154, Arg155, Trp156, His163, Ser170, Ser172, Gly175, Tyr210, and Tyr217 relative to SEQ ID NO:1 or SEQ ID NO:2. In some embodiments, such LGICs include five engineered LGIC subunits. In some embodiments, the engineered LGIC has an EC₅₀ of >20 μM for the ligand.

In yet another aspect, chemogenetic receptors that include an engineered LGIC as described herein are provided.

In still another aspect, cells that include an engineered LGIC as described herein or a chemogenetic receptor as described herein are provided. In some embodiments, the cell is a neuron. In some embodiments, the cell is in culture. In some embodiments, the cell is in vivo.

In another aspect, methods of treating a substance use disorder are provided. Such methods typically include delivering an engineered LGIC as described herein or a chemogenetic receptor as described herein to an individual, wherein, in the presence of the ligand, the engineered LGIC or the chemogenetic receptor reduces a reward response for the substance or increases an aversion response for the substance. In some embodiments, the engineered LGIC is delivered in the form of a nucleic acid encoding the engineered LGIC.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1C shows chemogenetics as a new mode of signaling control. FIG. 1A is a schematic showing that drug addiction is dependent on the complex interaction of behavioral and pharmacological properties of drug molecules. FIG. 1B is a graph showing that the route of drug administration strongly influences drug exposure level and time course (modified from Jones, 1990, NIDA Research Monograph, 99:30-41). FIG. 1C are graphs showing self-administration (SA) schedules that result in large “spikes” in drug exposure (top) lead to greater dependence than does continuous exposure (bottom) (modified from Zimmer et al., 2012, Neuropsychopharm., 37:1901-10).

FIG. 2A-2C demonstrates negative-feedback chemogenetics. FIGS. 2A and 2B show that chemogenetics can be used to engineer artificial negative feedback loops to control behaviors that are affected by the ingestion of an addictive drug that binds the chemogenetic receptor by targeting to specific neural circuit nodes. FIG. 2C show that chemogenetic rheostats (simulated data) can be used to reduce the activity of neurons that promote SA and to increase the activity of neurons that reduce SA (LHb: lateral habenula).

FIG. 3 is a schematic of PSAM chimeric ion channels. PSAMs developed from the ligand binding domain (LBD) of the α7 nAChR are spliced to either the IPD of 5HT3 or GlyR to produce chimeric channels for neuron activation or inhibition, respectively. PSAM-IPD subunits homo-pentamerize to form LGICs. The same PSAM and its cognate agonist (yellow circle) are used for both types of channel. Mutations in the LBD produce novel drug-agonists and reduce ACh sensitivity.

FIG. 4 shows the structural relationships of addictive drugs and nicotinic agonists. Structural elements that correspond to the nicotine pharmacophore are highlighted in red (amine protonated at physiological pH). Structural elements corresponding to the endogenous agonist acetylcholine are highlighted in blue. Drug classes are grouped together. The FDA-approved bupropion is a cathinone, which are also used in addictive street drugs referred to as ‘bath salts’.

FIG. 5 shows the crystal structure of the pentameric AChBP bound to cocaine (yellow) at the interface of two protomer subunits (cyan & pink). Some nearby amino acid side chains shown with numbering and labeling corresponding to the human α7 nAChR sequence (PBD:2PGZ from Hansen & Taylor, 2007, J. Mol. Biol., 369:895-901).

FIG. 6A-6C are schematics showing the approach for generating and optimizing chemogenetic receptors for addictive drugs. FIG. 6A is a schematic showing how a drug library can be screened against a library of mutated chimeric ion channels. ‘Hits’ are iteratively optimized by additional rounds of mutations in the LBD. Receptors for different drugs with distinct LBDs are identified (right). FIG. 6B is a schematic showing that putative chemogenetic receptors are characterized by electrophysiology in HEK-293 cells and cultured neurons. cDNA for suitable channels are packaged into AAV vectors for in vivo experiments. FIG. 6C is a schematic showing that chemogenetic receptors can be characterized by fiber photometry, neural circuit perturbations of intravenous self-administration (IVSA), and non-invasive in vivo pharmacology and functional activity.

FIG. 7A-7C show experimental data validating an ion channel screening assay. FIG. 7A is a dose response graph of α7-5HT3 expressed in HEK293 cells in a fluorescence membrane potential (MP) assay measured on the Hamamatsu FDSS liquid handling plate reader. FIG. 7B is a voltage clamp recording of current response from α7-5HT3 chimeric ion channel showing the peak current (Ipeak) and steady state current (Iss) in response to the agonist PNU-282987. FIG. 7C show dose response curves for α7-5HT3 from Ipeak, Iss, and MP assay maximum responses.

FIG. 8 shows a representative screen for chemogenetic cocaine receptors. Potency of cocaine and ACh agonism against a panel of ion channels with single-site mutations in and around the ligand binding domain of chimeric channels that include α7 nAChR LBD and IPD from either 5HT3 or GlyR. Red asterisks highlight chimeric channels with cocaine agonism and reduced ACh potency (receptors with no visible bar have EC₅₀>30 μM).

FIG. 9 is a graph showing an example of potency optimization (PSAM4-GlyR chimeric channel) for ACh and varenicline (see, e.g., Magnus et al., 2019, Science, eaav5282). Synergistic improvement of varenicline potency occurs when Leu131→Gly and Gln139→Leu mutations are combined. In addition, Tyr217→Phe reduces ACh potency. The numbers shown above the bars correspond to EC₅₀.

FIG. 10A-10I is experimental data showing the optimization of chemogenetic receptors for cocaine. FIG. 10A-10C show that Coca-5HT3 (FIG. 10A, 10B) and coca-GlyR (FIG. 10C) channel potency for cocaine (left) and ACh (right) compared to α7-5HT3 or α7-GlyR. Coca-5HT3 corresponds to α7-5HT3 with 4 mutations α7^{L141G,G175K,Y210F,Y217F}-(5HT3), where Leu141→Gly induces cocaine agonism, Gly175→Lys increases cocaine potency for the receptor, Tyr210→Phe and Tyr217→Phe both reduce ACh and choline potency. Coca-GlyR corresponds to α7-GlyR with 3 mutations (α7^{L141G,G175K,Y217F}-GlyR), where Leu141→Gly induces cocaine agonism, Gly175→Lys increases cocaine potency for the receptor, and Tyr217→Phe reduces ACh and choline potency. FIG. 10B shows the cocaine-activated currents from coca-5HT3 in HEK293 cells. Prolonged steady-state current. FIG. 10D is a graph showing the membrane properties in neurons expressing coca-5HT3 or coca-GlyR. FIG. 10E is a graph showing the depolarization magnitude in hippocampal neurons expressing coca-5HT3. FIG. 10F shows action potential firing in hippocampal neuron expressing coca-5HT3 in response to cocaine. Downward deflections are brief current injections to monitor membrane properties. FIG. 10G shows reduced excitability to depolarizing current injection in hippocampal neuron expressing coca-GlyR in presence of cocaine. FIG. 10H is a graph showing input resistance in response to cocaine for hippocampal neurons expressing coca-GlyR or GFP control. Recovery after cocaine removal (WASH). FIG. 10I is a graph showing the fold-change of current necessary to elicit an action potential (rheobase) in hippocampal neurons expressing coca-GlyR or GFP control.

FIG. 11. Experimental data showing that coca-5HT3 is selective for cocaine over cocaine metabolites (FIG. 11A), other addictive drugs (FIG. 11B), and amine-containing endogenous neuromodulator molecules (FIG. 11C).

FIG. 12A-B are graphs showing that neuron firing was increased in response to a synthetic Gpr40 agonist, GW9508 (0.2 μM) (FIG. 12A) or a fatty acid, palmitate (20 μM) (FIG. 12B).

### DETAILED DESCRIPTION

Addictive drugs and other ingested (e.g., orally, nasally) compounds act on brain circuits that are associated with diverse neurobiological processes affecting motivation, movement, wakefulness, and attention. The addictive properties of drugs and other ingested compounds are tied to their chemical properties and associated pharmacokinetics, which considerably impact reward and reinforcement. Pharmacological interventions for substance use disorder (SUD) have undesirable side effects because they influence general-purpose motivational processes as well as other behavioral and physiological systems. A challenge for SUD research is to modulate addiction-related neural circuits in a manner that models the time course of drug exposure, restricts neuromodulation to the drug-exposed state, and localizes interventions solely to circuits responsible for sustaining SUD.

Chemogenetics is a valuable neuroscience technique in which an exogeneous engineered receptor is expressed in a cell type of interest, where it is inert until engaged by a cognate chemical agonist, consequently resulting in small molecule-controlled neuromodulation. Chemogenetics is applied herein for drug- or other compound-controlled neuromodulation of the neural circuit pathways that mediate addiction. To investigate SUD, bespoke chemogenetic receptors are developed that are gated by addictive drugs. Such receptors can be used in a negative feedback process to investigate the cell types and circuits that can blunt drug addiction, either by reducing reward or increasing aversion solely during drug intake. Chemogenetic tools for neuron activation and inhibition have been developed based on chimeric ion channels constructed from the ligand binding domain of the α7 nicotinic acetylcholine receptor spliced to the ion pore domain of the cation-selective serotonin receptor or the anion-selective glycine receptor, respectively. As described herein, high-throughput screening methods are used to screen a library of chimeric ion channels with mutations around the canonical ligand binding site against a library of addictive drugs and their metabolites to identify channels that are gated by these drugs. As described herein, addictive drugs include, without limitation, cocaine, nicotine, amphetamines, cathinones, and opioids, from which many metabolites can be produced. Other ingested compounds whose ingestion would be understood to results in reinforcing behaviors include, for example, sugar, caffeine, and one or more fatty acids (e.g., 6-carbon to 18-carbon fatty acids (e.g., caproic acid, caprylic acid, capric acid, lauric acid, myristic acid, palmitic acid, and stearic acid)).

Once identified, such chemogenetic receptors can be further optimized, for example, by increasing potency for the addictive drug and/or reducing potency for endogenous agonists. The chemogenetic receptors for cocaine that are described herein were used to investigate the effectiveness of negative feedback chemogenetic control over the neural circuit pathways associated with motivation for cocaine self-administration. These neural circuit studies with cocaine can serve as a template for comparison of the pathways associated with other addictive drugs, such as nicotine, amphetamines, or opioids. The resulting chemogenetic receptors can allow investigation of mechanism to reduce drug self-administration in animal models (e.g., rodents) by selective negative-feedback neuromodulation to reduce drug-induced reward or to elicit drug-induced aversion. These studies will advance the understanding of neural control over drug seeking, and the chemogenetic receptors described herein (e.g., activated by addictive drugs) provide a potential avenue for new gene therapy approaches to selectively treat SUDs.

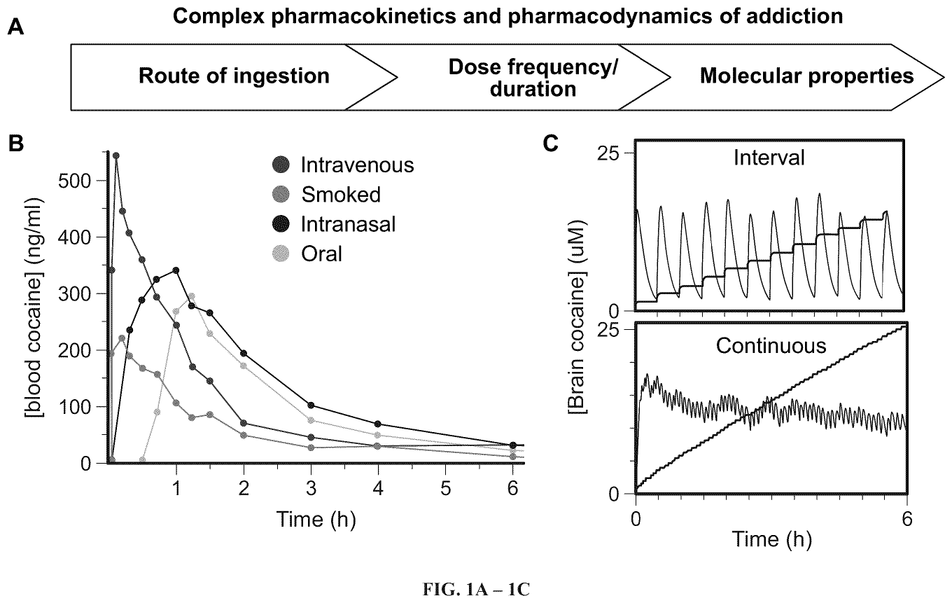

Multiple lines of evidence point to the importance of the temporal dynamics of drug exposure for effects on plasticity and behavior (FIG. 1A). First, the route of administration has a large influence on drug exposure pharmacokinetics (FIG. 1B), where rapid exposure to a drug bolus, for example by smoking or intravenous injection, leads more quickly to dependency and self-administration. For example, methylphenidate is more habit forming when administered intravenously or intranasally than orally, and abuse potential is further reduced with slow-onset controlled release. This principle underlies addiction therapies. Although methadone is habit-forming when delivered intravenously, oral methadone is used to treat opioid dependency. Similarly, controlled release formulation nicotine is used to counteract nicotine addiction, and sustained release amphetamine has shown potentially positive outcomes for treating cocaine dependence. Thus, the rapidity of drug exposure more strongly influences dependency than the area under the drug exposure curve.

In addition, the frequency and duration of drug administration can influence the onset of addiction, which has been noted for cocaine and nicotine. In many instances, the frequency and duration of drug administration is more important than the total amount of drug consumed (FIG. 1C).

Further, the intrinsic molecular characteristics of drugs (e.g., lipophilicity, PgP pump substrates) influence the time course of brain access, and this defines considerable differences in their reinforcing characteristics. For example, heroin is more rapidly taken up by the brain than morphine, due to greater lipophilicity.

Addiction hijacks general motivation and learning circuits. Drug addiction modulates circuits involving dopamine (DA) neurons, which influence multiple behavioral and physiological processes. DA neurons in the VTA are involved in reinforcement from natural rewards, such as food, water, and sex. Circuits that influence DA release or are modulated by DA are important for the habit-forming effects of addictive drugs, as well as other behavioral consequences on learning, sleep, movement, and attention. The importance of these circuits for essential behaviors is a challenge for drug addiction treatment due to the need to influence drug-seeking while minimizing consequences in other aspects of behavior and physiology.

In closed-loop experimental designs, the consequences of a behavior lead to a specific circuit perturbation to shape the temporal profile of neuromodulation to the specific behavior under investigation. To investigate the neurobiology of SUD while prioritizing the importance of the addictive drug with closed-loop perturbations, chemogenetic receptors were developed that are gated by addictive drugs, which ties neuromodulation to the precise time course of drug exposure. Past studies using chemogenetics and optogenetics to examine neural circuitry, even in the context of addiction, were open-loop experimental designs, where the drug addiction behavior and the chemogenetic perturbation were manipulated independently such that the neural circuit manipulation is not dependent on addictive drug pharmacokinetics.

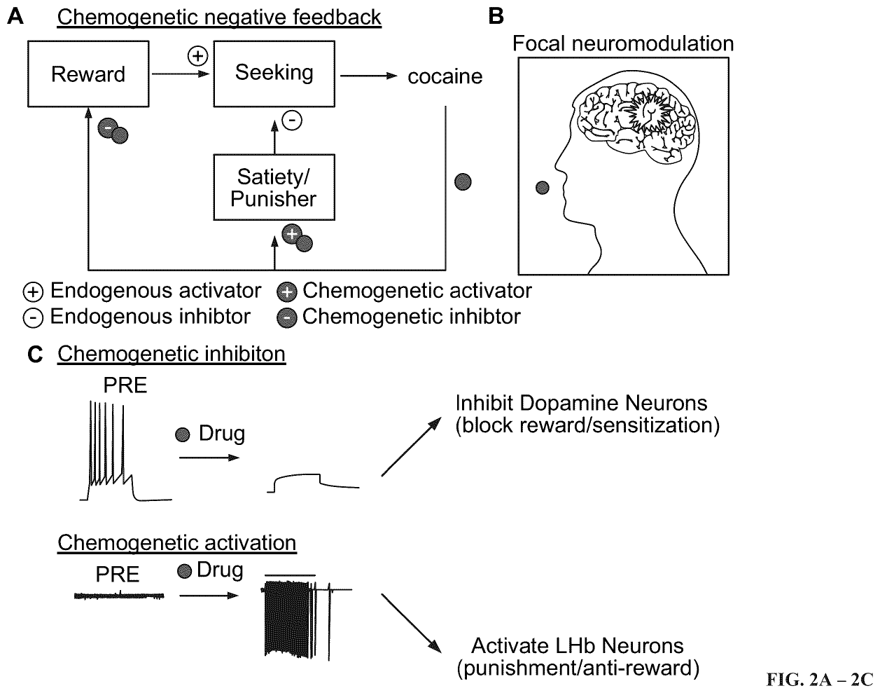

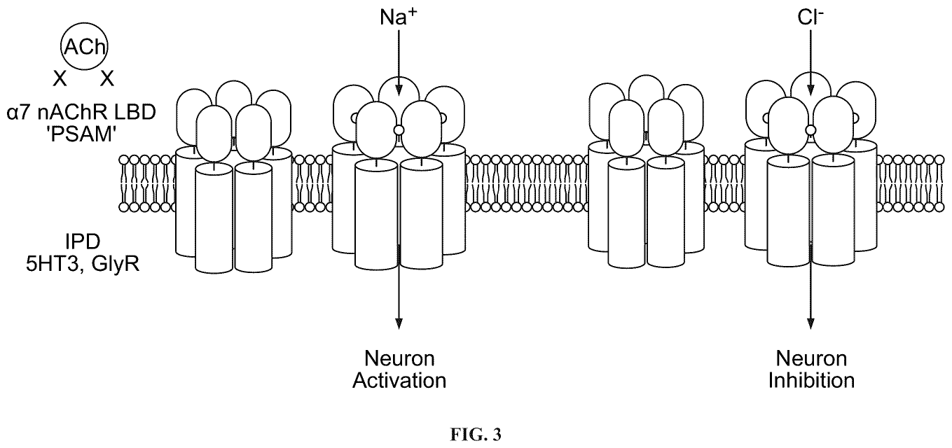

As described herein, drug-controlled chemogenetic receptors can be used in a negative feedback process to investigate the cell types and circuits that can blunt addictive potential for certain substances, either by reducing reward or increasing aversion (FIG. 2A, 2B). There is clinical precedence for both strategies to reduce drug seeking. Opioid addiction treatment uses long-lasting opioid receptor agonists to blunt the reward and reinforcement effects of highly addictive opioid drugs. As an alternative strategy, the anti-alcohol abuse treatment drug, disulfiram (Antabuse), antagonizes the enzyme alcohol dehydrogenase, resulting in an aversive state after alcohol ingestion that leads to negative association. Negative feedback chemogenetics could be deployed in an analogous manner to investigate the contribution to addiction behaviors of virtually any neural population in the body (FIG. 2C). As indicated above, the advantage of closed-loop chemogenetics is that pharmacological regulation is strictly tied to the time course of drug intake; therefore, only the drug to which the chemogenetic receptor is responsive affects signaling, and signaling during ingestion of natural rewards is unaffected. Moreover, a chemogenetic receptor can be localized to specific neural circuit nodes using viral or genome engineering methods (FIG. 2B), whereas pharmacological anti-addiction treatments influence targets throughout the brain and the body.

There are two main platforms for modular chemogenetics in neurons: G-protein coupled receptors (GPCRs) and ligand gated ion channels (LGICs). One example of GPCRs are DREADDs, which are developed by directed evolution followed by extensive optimization to produce neuron activation or silencing using clozapine-N-oxide. The mechanisms-of-action for DREADDs are functionally similar to muscarine for neuron activation and baclofen for neuronal inhibition and are dependent on the effectiveness of the G-protein signaling pathways coupling to a specific set of ion channels that must be present in the targeted cell type. LGICs can be used for direct pharmacological control over ion conductance. The functional properties of ion channels are primarily dictated by their ion selectivity. Inward flux of cations or outward flux of anions depolarizes cells, and correspondingly inward flux of anions generally leads to reduced neuron activity. The mechanisms of action are functionally similar to nicotine for neuron activation and muscimol for neuronal inhibition.

Accordingly, a number of chemogenetic receptors are described herein that bind a molecule and modulate the function of a cell, where the molecule is a type that reinforces its own ingestion or administration (e.g., an addictive drug) and the cellular modulation occurs upon binding of the molecule. For example, the following chemogenetic receptors are provided herein: cocaine-LGIC; cocaine-GPCR; MDMA-LGIC; MDMA-GPCR; heroin-LGIC; heroin-GPCR; nicotine-LGIC; nicotine-GPCR; oxycodone-LGIC; oxycodone-GPCR; morphine-LGIC; morphine-GPCR; fentanyl-LGIC; fentanyl-GPCR; fatty acid-LGIC; and fatty acid-GPCR.

A modular chemogenetic platform from chimeric LGICs derived from α7 nicotinic acetylcholine receptor (nAChR) and other Cys-loop family members is available (Magnus et al., 2019, Science, eaav5282; Magnus et al., 2011, Science, 333:1292-6). The extracellular ligand binding domain (LBD) of α7 nAChR is transferrable to the transmembrane ion pore domains (IPDs) of other members of the Cys-loop LGIC family (FIG. 3). This property allows the pharmacology of the LBD component to be maintained while accessing the ion conductance properties of other LGICs, such as the cation-selective serotonin receptor 3 (5HT3) or the anion-selective glycine receptor (GlyR) to generate chimeric channels. These “α7-5HT3” or “α7-GlyR” chimeric channels respond to nicotinic agonists but have the ion conductance and the steady-state currents (e.g., only partial desensitization) of the 5HT3-R and the GlyR.

Different mutations in the α7 nAChR LBD confer different selective agonist activity for structurally distinct small molecules while reducing endogenous agonist potency of acetylcholine (ACh). The mutated α7 nAChR LBDs were termed pharmacologically selective actuator modules (PSAM, pronounced “sam”). Each PSAM can be spliced to a different IPD to achieve novel pharmacological control over distinct ion conductances. PSAM-5HT3 channels provide prolonged depolarizing currents in the presence of the corresponding agonist, leading to sustained neuron activation. PSAM-GlyR channels have large chloride-selective conductance with a long steady state window current to maintain silencing as long as the agonist is present. Different PSAM LBDs have been developed that each confer selectivity to novel molecules (i.e., not nicotinic agonists), as well as clinically used drugs, e.g., tropisetron (anti-emetic), granisetron (anti-emetic), varenicline (anti-smoking drug).

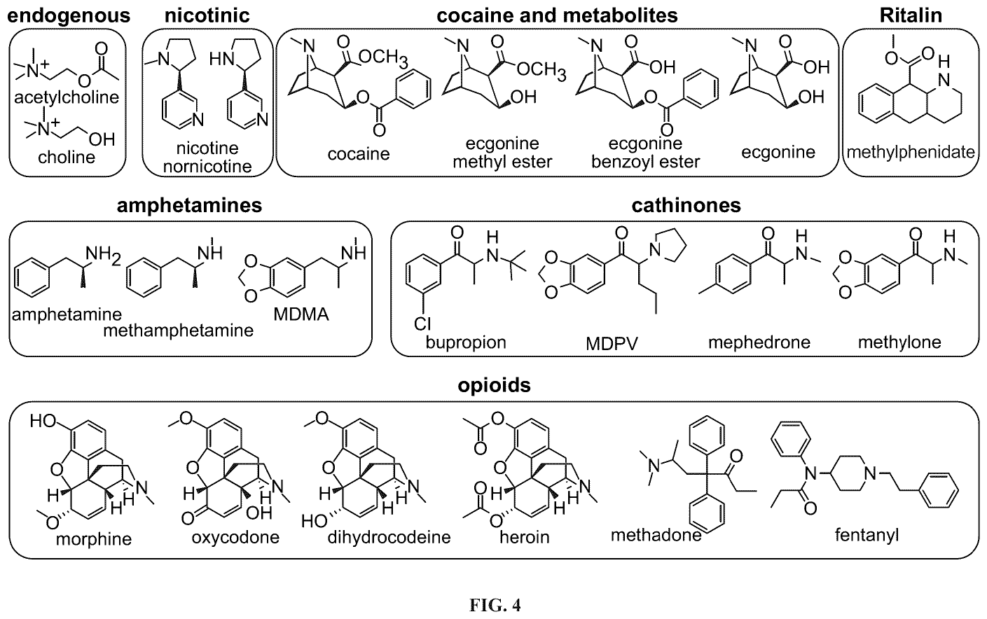

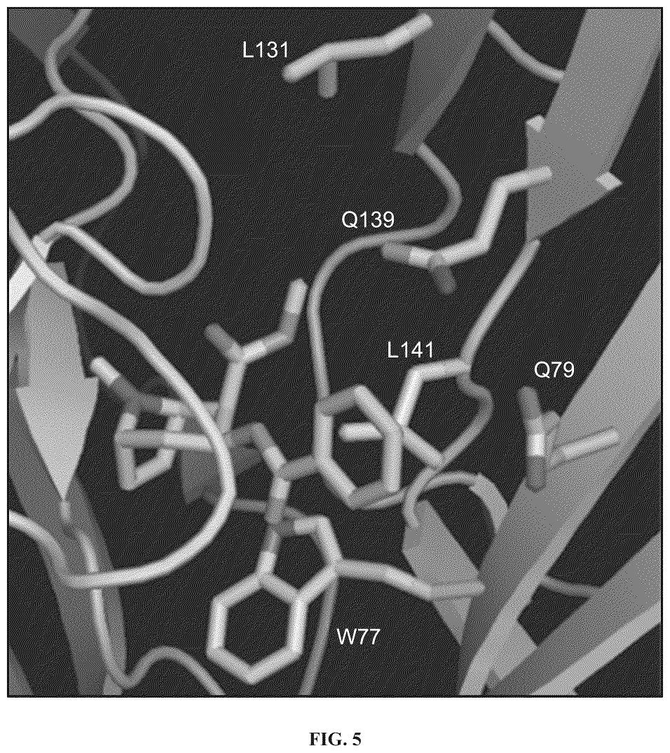

The α7 nAChR LBD offers an ideal foundation for chemogenetic receptors activated by addictive drugs because ligands for nAChRs have pharmacophores with structural similarity to many addictive drugs such as nicotine as well as cocaine, amphetamines, cathinones, and opioids (FIG. 4). In addition, many addictive drugs bind nAChRs as antagonists. To generate chemogenetic receptors for these compounds involves modifying the ligand binding site to promote agonist activity of these antagonists. Also, it is important to match chemogenetic receptor potency to the activity of an addictive drug at its endogenous pharmacological target. For example, the agonist EC50 for the chemogenetic receptor should be less than the maximal drug exposure concentration during drug-taking.

Cocaine inhibits the dopamine transporter (DAT), which is responsible for its addictive effects. Cocaine is also a low to moderate affinity antagonist of multiple nAChR subtypes. Furthermore, there is a crystal structure of the homologous acetylcholine binding protein (AChBP) bound to cocaine, which shows a binding pose similar to nicotine (FIG. 5), with the important difference that the cocaine tertiary amine is offset from a key backbone H-bond with Trp171 that is characteristic of nicotine, which likely accounts for lack of cocaine agonist activity. Alteration of the amino acid residues in α7 nAChR LBD surrounding cocaine can alter its binding pose in order to convert cocaine to an agonist for a chemogenetic receptor. It is also important to examine chemogenetic activation by the metabolites of cocaine such as benzoyl ecgonine and ecgonine, which retain the tropane pharmacophore of cocaine that is associated with nAChR binding. For chemogenetic perturbations that recapitulate the timecourse of cocaine, it is preferable to limit activation by these long-lived metabolites, although there may be chemogenetic applications for these ligands where a prolonged chemogenetic neuromodulation during and/or following drug-taking is desired.

Cocaine inhibits DAT with reported Ki ranging from 0.23-2.0 μM, providing a lower bound for chemogenetic receptor EC50 (i.e., potency). However, the cocaine brain concentration maintained by self-administration in rat is estimated to be ˜10-20 μM, defining an upper bound. To clarify the sensitivity of drug-seeking to neuronal perturbation at different cocaine exposure concentrations, different chemogenetic receptors with potency tiling the range of 10 μM>EC50coc>0.2 μM would be valuable for drug-gated neuromodulation, which is a typical aspect of chemogenetic receptor optimization.

Methylphenidate is an inhibitor of DAT and can lead to self-administration in rodents and a “high” feeling in humans following intravenous dosing. The EC50 for methylphenidate-mediated release of DA in the striatum is 10 μM, thus EC50<10 μM is suitable for a methylphenidate chemogenetic receptor.

Nicotine activates nicotinic receptors on VTA dopamine neurons, leading to dopamine release in the nucleus accumbens with EC50: 0.48 μM. Nicotine agonist activity at α7-5HT3 and α7-GlyR (nicotine: EC50α7-5HT3: 6.7 μM, EC50α7-GlyR: 6.8 μM) need to be improved for nicotine chemogenetic activation ˜10-70-fold (EC50: 0.1-0.5 μM), while reducing potency of endogenous agonists (ACh: EC50 α7-5HT3: 8.1 μM, EC50 α7-GlyR: 6.4 μM; Choline: EC50 α7-5HT3: 37 μM, EC50 α7-GlyR: 103 μM). In addition, nornicotine is a nicotine metabolite to be evaluated for chemogenetic receptor binding.

Amphetamine and methamphetamine reduce DAT function, leading to elevated dopamine release from dopamine release sites. Amphetamine also binds to α7 nAChR as an antagonist, which is consistent with the presence of an accessible amine functional group, structurally related to ACh (FIG. 4). Docking studies with ACh-BP indicated that antagonist activity of amphetamine was due to an amine-protein hydrogen bond with the carbonyl backbone of Ser170, whereas most agonists show a hydrogen bond interaction with the backbone carbonyl of Trp171. Alterations to the steric environment around Ser170 that disfavors this H-bond might shift binding to a putative agonist-mode at Trp171. During self-administration, blood methamphetamine reaches 0.7 μM (in males) to 0.9 μM (in females), whereas the amphetamine EC50 for DA release is 0.5 μM. Thus, amphetamine and methamphetamine chemogenetic receptors have EC50: 0.5-1 μM.

Bupropion has been shown to inhibit α7 nAChR with weak affinity. Bupropion is structurally similar to addictive drugs called cathinones, which are also known by the street name ‘bath salts’. Cathinones include methylenedioxypyrvalerone (MDPV) as a major component, which supports self-administration (EC50 MDPV: 0.1 μM for dopamine released). Mephedrone and methylone (EC50 mephedrone: ˜0.3 μM, EC50 methylone: ˜0.4 μM) are additional cathinone components of bath salts. Thus, corresponding chemogenetic receptors have EC50: 0.1-0.4 μM.

Opioid dependency is associated with binding the mu-opioid receptor, which leads to disinhibition (i.e., activation) of VTA dopamine neurons. Morphine also binds and inhibits α7 nAChR. A crystal structure of the AChBP shows galantamine, which is structurally similar to morphine, in the ligand binding site near the backbone carbonyl corresponding to Trp171 in the α7nAChR sequence. The structural similarity between morphine, heroin, and oxycodone makes each of these candidate agonists for chemogenetic receptors: oxycodone (EC50 MOR: 1.4 μM), hydrocodone (EC50 MOR: 1.5 μM), morphine (EC50 MOR: 0.19 μM), methadone (EC50 MOR: 0.04 μM), fentanyl (EC50 MOR: 0.01 μM). Chemogenetic receptors activated by opioids with these potencies are useful for addiction research applications.

The addictive-drug target potencies listed above are within the range for previously discovered PSAMs that are activated by other molecules. Other drugs, however, with distinct pharmacophores (e.g., cannabinoids, barbiturates, and benzodiazepines) are likely not good candidates for developing chemogenetic receptors using α7 nAChR LBD.

The pharmacological specificity of chimeric ion channel chemogenetic receptors is determined by the α7 nAChR LBD. Like the parent chimeric channel, α7-5HT3, for neuron activation, a PSAM with α7 nAChR LBD mutations is spliced onto the 5HT3 IPD, which leads to inward flux of cations and neuronal depolarization. The 5HT3-R has low single channel conductance, which has been found to be suitable for avoiding depolarization block during neuron activation that can be observed with high conductance ion channels.

For neuron inhibition, the α7 nAChR can be spliced onto chloride-selective IPDs from either the glycine receptor (GlyR) or the GABA C receptor (GABAR). These are high conductance channels, where relatively low expression levels are needed to suppress neuron firing. Chloride channel activation is widely used to inhibit neuron activity though several mechanisms: 1) opening high conductance ion channels electrically shunts the cell membrane; 2) some neurons are hyperpolarized by chloride channels, which moves the cell further from action potential threshold; 3) PSAM-GlyR chemogenetic chloride channels can suppress axonal transmission and block neurotransmitter release; and 4) some neurons are depolarized by chloride channels, which can inactivate voltage-gated chloride channels, thereby reducing excitability. An exception to this is striatal medium spiny neurons (MSNs), which fire action potentials in response to GABA and GABA-R agonists, due to an unusually low resting membrane conductance and membrane potential below the reversal potential for chloride. More generally, if neurons are empirically found to be inhibited by muscimol or GABA, then they will be inhibited by PSAM-GlyR chemogenetic ion channels, which is the case with most neurons that have been examined for appetite, motor function, learning, blood pressure, and pain.

The role of addictive substances for reinforcement and learning is essential for drug-seeking behaviors and involves elevated dopamine release from the ventral tegmental area (VTA) into the nucleus accumbens (NAc), prefrontal cortex (PFC), and amygdala. Addictive drugs directly or indirectly modulate dopamine levels throughout the brain, which has extensive consequences throughout the brain and body. Nicotine directly depolarizes dopamine neurons, eliciting increased neuron firing and dopamine release. Opioids suppress inhibitory tone onto dopamine neurons, indirectly leading to increased dopamine neuron activity and release. Additional molecules, such as cocaine, methylphenidate, amphetamines, and cathinones suppress or reverse the dopamine reuptake process at synaptic terminals in the NAc, leading to elevated extracellular dopamine.

As described herein, negative-feedback chemogenetics can reduce the reward/reinforcement processes modulated by addictive drugs or activating aversive circuits to suppress drug-seeking. Chemogenetic receptors can be delivered to specific neural populations using adeno-associated viral (AAV) vectors or other methods. For example, chemogenetic inhibition of dopamine neurons can suppress an essential circuit node for addiction. This is expected to be most effective for drugs that act to increase dopamine neuron activity, such as nicotine or opioids. It is less clear if dopamine neuron somatic inhibition would be effective for reducing self-administration of cocaine and amphetamines, which act at the dopamine neuron release site. Nevertheless, past work indicates that PSAM-GlyR can suppress axonal activity and synaptic release.

The lateral habenula (LHb) is another circuit node relevant for addiction behaviors and is associated with negative affect. Neurons in the LHb increase activity in response to missed rewards or aversive outcomes, and LHb receives VTA input and activation is aversive, leading to avoidance behaviors. Cocaine initially suppresses LHb after drug-taking, and 24 h following self-administration, LHb neurons show elevated excitability, which is potentially indicative of a role in withdrawal or negative mood. Thus, LHb activation represents an approach to impose a cost on drug-taking. In addition, the LHb has been reported to blunt excessive reward activation (‘anti-reward’) via the RMTg, where a LHb GLUTAMATE→RMTgGABA→VTA-DA circuit inhibits VTA dopamine neurons. The LHb also projects to other motivationally relevant brain regions controlling serotonin release and the neuroendocrine axis.

Chemogenetic inhibition of VTA-DA neurons and chemogenetic activation of LHb neurons have significant potential to modulate drug-seeking behavior in a manner dependent on the drug mechanism-of-action. Thus, these circuits are well-suited for initial validation of negative-feedback chemogenetics.

Evaluation of in vivo chemogenetic receptor pharmacology and neural network activation. Similar to other chemogenetic technologies, translational and potential clinical applications of negative-feedback chemogenetics would necessitate noninvasive and longitudinal monitoring of the receptor's location and expression, its level of drug engagement (i.e., receptor occupancy), and its effect on global neural network activity. Positron emission tomography (PET) is a translational molecular imaging modality that is uniquely suited to address these requirements and facilitate both development of bespoke chemogenetic receptors and their in vivo application. Furthermore, PET can be combined with behavioral procedures to establish effects of chemogenetic modulation on neural network activity concurrent with effects on behavior.

SUD can be extremely difficult to overcome, despite a strong motivation to quit. It is often difficult to entirely remove an individual from environmental triggers that lead to relapsed drug-taking. Gene therapies involve administering the coding sequence for an exogenous protein to specific cell populations, and this approach is increasingly utilized for chronic diseases that are resistant to other therapeutic approaches. Although negative-feedback chemogenetics is a tool for investigating the neural mechanisms of drug addiction, negative-feedback chemogenetics also is a potential approach to pharmacotherapy-resistant SUD. For negative-feedback chemogenetics, the chemogenetic receptor would be delivered to a circuit node associated with reducing drug-seeking in order to engage it selectively during drug consumption but not in response to other rewards. Importantly, negative-feedback chemogenetics would not interfere with behavioral and environmental therapy because it is non-perturbative in the absence of drug-taking.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

The experiments described herein involve identifying mutant chimeric ion channels for different addictive substances, optimizing these ion channels as chemogenetic receptors for mammalian brains, characterizing their functional activity, and demonstrating their effectiveness for suppressing drug self-administration by negative-feedback chemogenetics.

### Example 1—Library of α7 nAChR LBD Mutant Chimeric Ion Channels and Addictive Drug Panel

Chimeric ion channels constructed using the α7 nAChR LBD form homomeric pentamers with the agonist binding site at the interface between promoter subunits. Engineering new pharmacological responsiveness into these channels is most readily achieved by mutating amino acid residues surrounding the agonist binding site. The crystal structure of the homologous AChBP bound to nicotine and other molecules provides guidance about nearby residues. In past work, a library of amino acid substitutions at α7 nAChR LBD residues Trp⁷⁷, Gln⁷⁹, Gln¹³⁹, and Leu¹⁴¹ was generated (Magnus et al., 2019, Science, eaav5282; Magnus et al., 2011, Science, 333:1292-6). This library of ion channels formed a starting point for identifying multiple chemogenetic receptors. This set of mutated chimeric ion channels is expanded to include additional residues in the vicinity of the agonist binding site (e.g., Ser⁵⁶, Ser⁵⁸, Leu¹¹³, Tyr¹¹⁵, Ser¹¹⁷, Leu¹³¹, Ser¹⁷⁰, Ser¹⁷², Tyr²¹⁷).

Mutated Ion Channels. A library of mutations in the α7-5HT3 chimeric cation channel is generated. An initial library of α7-5HT3 chimeric ion channels with mutations at Trp⁷⁷ (3), Gln⁷⁹ (12), Gln¹³⁹ (16), Leu¹⁴¹ (12) was used, where the numbers in parentheses are the number of mutant ion channels for each position. This is less than the possible 19 amino acid mutations, which is based on using channels with established cell-surface expression. Analysis of drug-bound crystal structures of AChBP indicates that amino acid residues Ser⁵⁶, Ser⁵⁸, Leu¹¹³, Tyr¹¹⁵, Ser¹¹⁷, Leu¹³¹, Ser¹⁷⁰, Ser¹⁷², Tyr²¹⁷ should be mutated to all other 19 amino acids. These mutants are initially screened for cell surface expression by binding Alexa594-labeled bungarotoxin in 6-well plates, followed by fluorescence microscopy, as in previous work (Magnus et al., 2011, Science, 333:1292-6). Based on previous work, 80% of mutated channels show cell surface expression and are used for drug screening. In total, a library of 277 (9 new residues*19 amino acids*0.8 cell surface rate+44 existing single mutant+96 existing multiple mutant) chimeric cation channels are generated with high binding site diversity for screening.

Drug Panel. A panel of 19 addictive drugs and their metabolites plus the two endogenous agonists, ACh and choline (Table 1) are generated. Many drugs and metabolites are available in small quantities as drug standards in organic solvent. Stock solutions are generated by purchasing drug standards, evaporating the solvent, and dissolving in DMSO as 100 mM stock solutions suitable for screening (final maximum DMSO concentration: 0.03%). The remaining compounds are purchased as neat compounds or salts (pure solids or liquids) and dissolved in DMSO (100 mM) for screening. Stock solutions are stored at −20° C. in glass vials and allowed to warm to room temperature before opening.

TABLE 1
Drug Library

| Drug/Metabolite/Endogenous | Type | Source |
|---|---|---|
| ACh | Endogenous agonist | Sigma |
| Choline | Endogenous agonist | Sigma |
| Nicotine | Nicotine | Sigma |
| Nornicotine | Nicotine metabolite | Sigma |
| Cocaine | Cocaine | Sigma |
| Benzoylecgonine | Cocaine metabolite | Sigma (B4147) |
| Ecgonine methyl ester | Cocaine metabolite | Sigma (E-001) |
| Ecgonine | Cocaine metabolite | Sigma (E-004) |
| Amphetamine | Amphetamine | Sigma |
| Methamphetamine | Amphetamine | Sigma(1399001) |
| MDMA (Ecstasy) | Amphetamine | Sigma (M-013) |
| Methylphenidate | Ritalin | Sigma (M-083) |
| Methylenedioxypyrovalerone | Cathinone (bath salts) | Sigma (M-146) |
| Mephedrone | Cathinone (bath salts) | Sigma (M349) |
| Methylone | Cathinone (bath salts) | Sigma (M-140) |
| Morphine | Opioid | Sigma |
| Oxycodone | Opioid | Sigma (O-002) |
| Heroin | Opioid | Sigma (H-038) |
| Fentanyl | Opioid | Sigma (F-013) |
| Methadone | Opioid | Sigma (M-007) |
| Hydrocodone | Opioid | Sigma (H-003) |

### Example 2—High Throughput Screening for Chemogenetic Receptors Against a Library of Addictive Drugs, Metabolites, and Endogenous Agonists

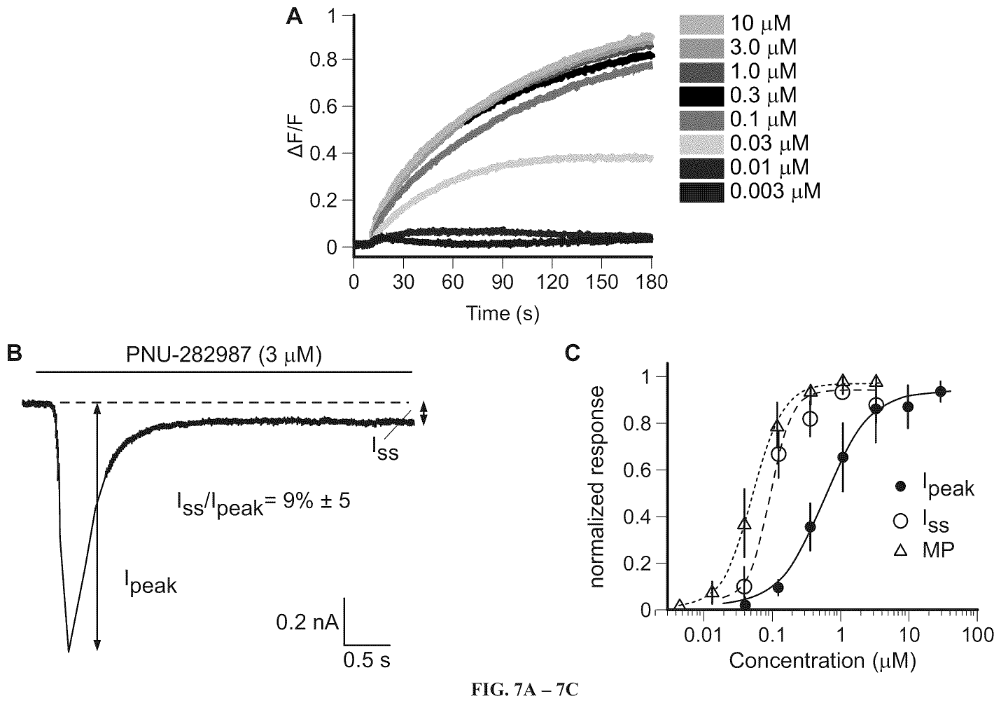

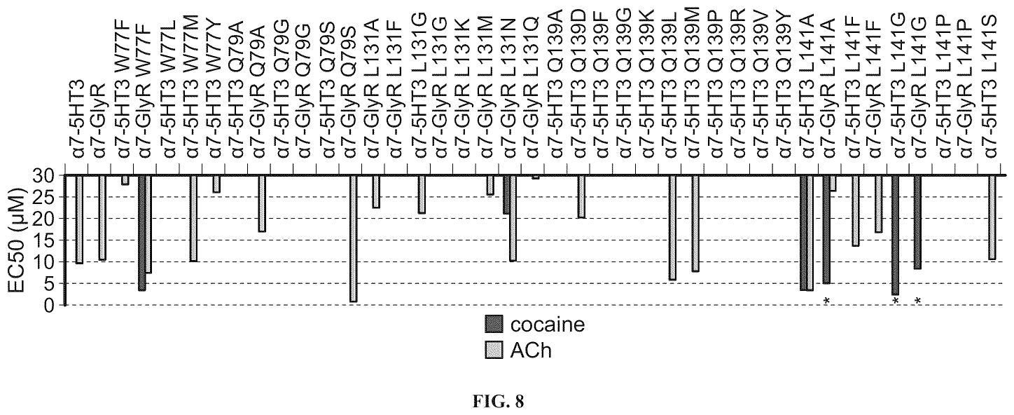

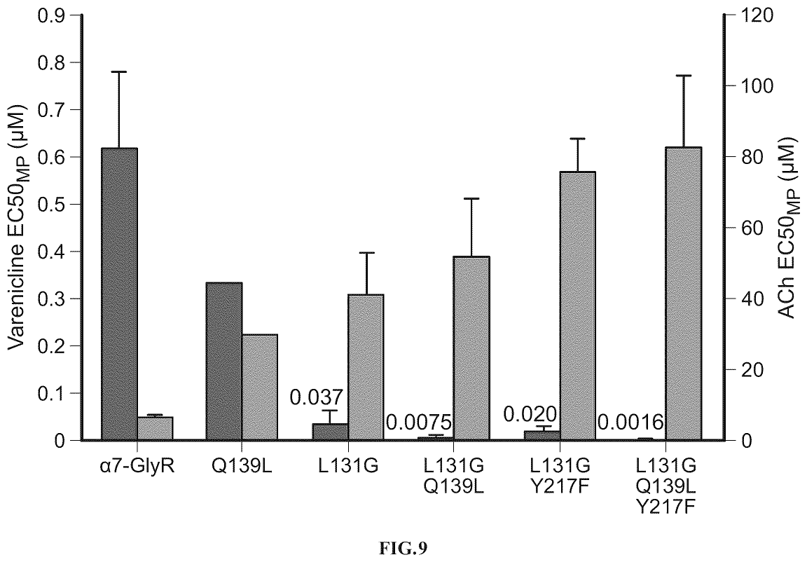

For chemogenetic receptor discovery, a small molecule is selected and a library of potential receptor mutants are screened against that small molecule. Transient transfection of putative chimeric ion channels in HEK cells is suitable for dose response screening. A 96-well plate-compatible fluorescence membrane potential (MP) assay was used to screen libraries of mutant chimeric ion channels against small molecules (FIG. 7A). The MP assay yields dose responses that reflect sustained (steady-state) channel activation (FIG. 7B), which is most relevant for chemogenetic applications. This assay has been used for 15 years to screen receptors, and it has been consistently found that the 50% effective concentration in this assay (EC50_{MP}) corresponds to the steady state current (EC50_{SS}) from electrophysiological recordings in HEK cells expressing the same channel, whereas the large peak current response (EC50ₚₑₐₖ) is right-shifted relative to EC50_{MP} (FIG. 7C).

In preliminary studies, 40 chemogenetic receptors with single amino acid substitutions at 5 positions were screened from the existing library of chimeric ion channel constructs. These ion channels have been previously curated for those that express in HEK293 cells. The ion channels were tested using dose responses for cocaine and its metabolites, benzoyl ecgonine and ecgonine, as well as the endogenous agonists, ACh and choline (FIG. 8). Chimeric ion channels with LBD amino acid substitutions that are in proximity to cocaine (Trp⁷⁷→Phe, Leu¹³¹→Asn, and Leu¹³¹ →Ala) showed some cocaine agonist activity but had undesired activity with the endogenous agonist, ACh. Substitution of Leu¹⁴¹→Gly produced a striking induction of cocaine agonism and exhibited reduced ACh potency. The bulky side chain of Leu¹⁴¹ is predicted to be proximal to the carboxymethyl ester of cocaine based on AChBP (see, e.g., FIG. 5), indicating that reduction of steric clash by substitution with Gly in the ligand binding may facilitate cocaine binding in an orientation suitable for agonist activity.

Specifically, a plate reader-based fluorescence membrane potential assay is used to screen the library of 277 mutated chimeric ion channels against a library of drugs with 21 related pharmacophores, including stimulants, opioids, and some of their primary metabolites. Including pharmacological positive controls, 6,648 dose response curves are generated in this primary library-against-library screen.

Chemogenetic receptors are tested after transient transfection of HEK-293 cells (Fugene HD). Dose response curves are robust to batch-to-batch differences in transfection. For each chemogenetic receptor, transfected cells are delivered to two 96-well Poly-D-Lysine coated black/clear cellware with a cell density of 0.6e06 live cells in 200 μL per well. Cells are incubated (22-26 h, 37° C. in 8% CO₂), then media is aspirated, and the membrane potential assay solution (Molecular Devices #R8034) is added. Drug dilution plates are prepared prior to the assay. For 21 compounds, two 96-well polypropylene sample plates are used containing dilutions down each column to achieve an 8-point dose response. ACh, choline, and nicotine are replicated in both plates as positive controls (total of 24 dose responses/receptor). Addictive drug and metabolite screening concentrations are tested at half-log intervals (3-fold dilution) from 0.01 μM to 30 μM and endogenous agonists are tested from 0.03 μM to 100 μM. To measure ion channel activation, a Hamamatsu FDSS 6000 plate reader and liquid handling system is used. The assay plates are scanned at 1 Hz (excitation: 472/30 nm, emission: 540/40 nm).

Compound solutions (50 μL) are simultaneously delivered from a 96 well compound plate after 10 baseline scans that are followed by 170 additional scans (180 s total). To calculate EC50_{MP}, the maximum response for each well is extracted, normalized to the maximum response for each compound, and sigmoidal dose response curves are calculated using software written in Matlab (Mathworks Central: ec50.m by Carlos Evangelista v1.0, Jan. 7, 2004) and the nlinfit function from the Statistics Toolbox. Chimeric ion channel-drug combinations with EC50_{MP}<10 μM are retested and advanced for optimization as chemogenetic receptors.

Based on screening data, different mutated ion channels have selectivity for a particular drug class (e.g., it is not necessarily the case that a single chemogenetic receptor that binds all tested drugs will be identified). Instead, representative receptors for each class of drugs (i.e., nicotine, cocaine, amphetamines, cathinones (“bath salts”), and opioids) are identified. In the screen, addictive drugs with a nicotine pharmacophore are most likely to show chemogenetic receptor hits with suitable potency, e.g., nicotine, cocaine, methylphenidate, MDPV, and most opioids (see, e.g., FIG. 4). Amphetamines, most cathinones, and methadone have a more departure from the quaternary-amine pharmacophore of acetylcholine, requiring discovery of an LBD that positions the 1°- or 2°-amines of these molecules to be compatible with agonist activity. Importantly, a cocaine-chemogenetic receptor was identified, and ion channel agonism by the amphetamine derivative, MDMA, as well as the opioid, fentanyl, has been observed, showing that these pharmacophores are able to produce chimeric ion channel agonists. Moreover, nicotine is an established agonist of these channels and the goal is to improve potency to a useful physiological range while reducing endogenous agonist sensitivity.

### Example 3—Mutagenesis to Optimize Drug Agonist Potency

Unlike traditional drug screening, the ‘hits’ in the present screen are mutant ion channels. Chemogenetic receptors with agonist activity for a drug are retested in replicates of three or greater. The receptors are then characterized by whole cell electrophysiology in transiently transfected HEK cells (FIG. 7B) to confirm EC50 and sustained steady-state channel opening with a ligand application of 1 min.

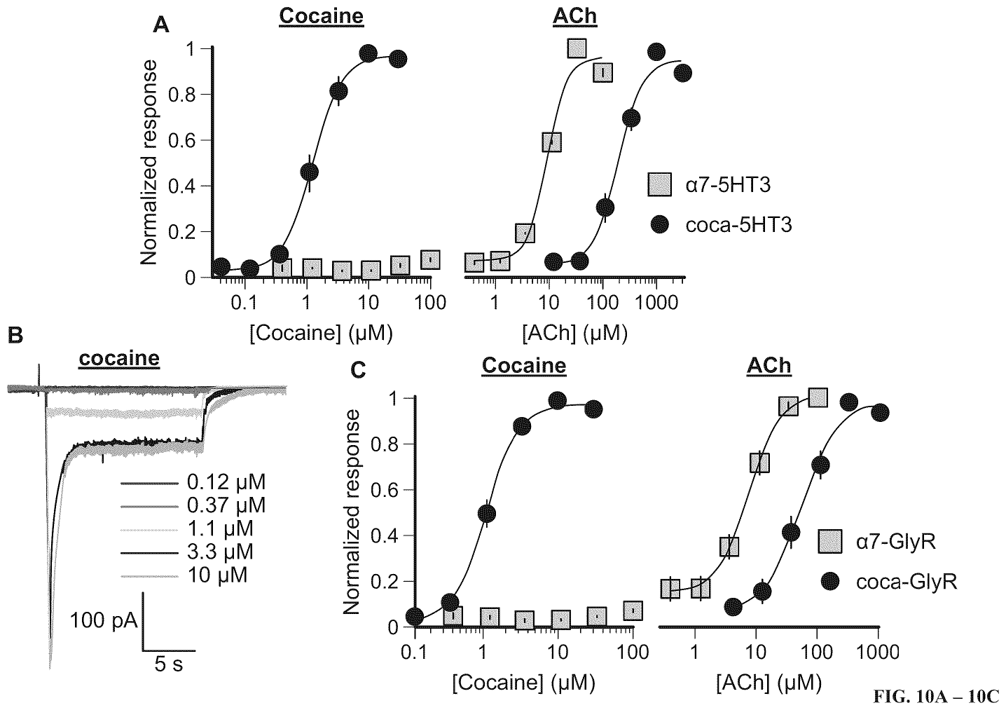

For chemogenetic applications in vivo, additional optimization of the LBD is needed to further improve drug potency and reduce endogenous agonist potency. For this, we take advantage of a property of α7 nAChR that dissociates the activity of exogenous drugs and endogenous agonists, based on different binding orientations. Most exogenous agonists (e.g., nicotine) form a H-bond with the backbone carbonyl of Trp¹⁷¹, while the endogenous agonists ACh and choline bind in a different configuration, relying on cation-pi interactions of the quaternary amine group with the numerous Tyr residues in the binding site. We have found that there are 3 types of mutations that are generally useful for optimizing chimeric channel pharmacology. Type 1: Synergy between two mutations identified in the screen that gave moderate potency improvement. Type 2: The activity of endogenous agonists ACh and choline is reduced by mutation of either Tyr¹¹⁵, Tyr²¹⁰, Tyr²¹⁷ to Phe, oftentimes with limited potency reduction for drug agonists. Type 3: Mutation of Gly¹⁷⁵→Lys improves agonist potency selectively for nicotinic-type agonists relative to ACh. An example of the optimization process using mutations of Type 1 and 2 is shown in FIG. 9 from development of PSAM⁴-GlyR, a chemogenetic receptor for varenicline.

Distinct combinations of Type 1, 2, and 3 mutations are introduced to improve agonist potency of drugs and reduce potency of endogenous agonists, ACh and choline. Past experience has shown that this requires generating ˜30 multiple mutation (3-7) receptors, which are tested against all 21 drugs to assess improvement for the primary drug as well as to identify selectivity against other drugs.

Receptors containing combinations of mutations are tested iteratively to improve or maintain drug potency while shifting ACh potency. Drug potencies are optimized to the levels described previously. To remove sensitivity to physiological concentrations of endogenous agonists, the goal for ACh potency is at least >20 μM and a preferred potency of >100 μM.

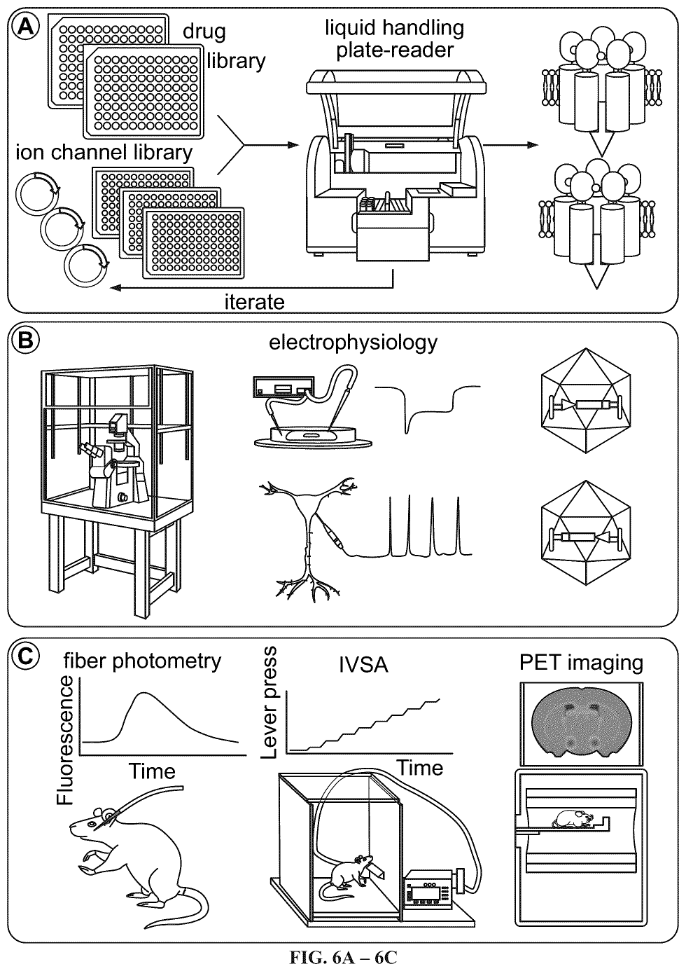

The LBD mutations from hits that meet the screening benchmarks also are used to generate the corresponding PSAM-GlyR channels for neuron inhibition simply by introducing the same LDB mutations in the α7-GlyR chimeric channel. Both excitatory and inhibitory channels can be accurately evaluated using the plate-reader-based fluorescence MP assay. Subsequently, these channels are tested using whole cell electrophysiology in transiently transfected HEK cells (see, e.g., FIG. 6B). Correspondence of the steady state EC₅₀ with EC50_{MP} are verified for the drug agonist as well as endogenous agonists. Channels with ligand-independent channel current (leaky channels) are eliminated, which is revealed by large holding currents in voltage clamp recordings. Channels with small whole cell currents (likely limited membrane trafficking) or complete desensitization with exposure to agonist (rarely observed with these chimeric ion channels, unlike native α7 nAChR) are not considered. Binding constants of drugs for chemogenetic receptors are determined by displacement of [³H]-ASEM or the associated radiolabeled drug, depending on availability.

A chemogenetic receptor for neuron activation by cocaine, coca-5HT3, has met the design benchmarks for potency and selectivity. This receptor is used for negative feedback chemogenetic investigation of addiction circuitry. The potency for cocaine is improved in additional channels using the Leu¹⁴¹→Gly mutation around the lower range for reported DAT Kᵢ values (EC₅₀: 0.3 μM) to ensure chemogenetic modulation that fully corresponds to endogenous cocaine pharmacodynamics.

The potency benchmarks for drugs associated with higher physiological peak concentrations for physiological effects (e.g., cocaine, methylphenidate, oxycodone, hydrocodone) is achievable because higher EC₅₀ values are readily discovered with chemogenetic receptors. Chemogenetic receptors with potencies from 0.1-1 μM also are typically achievable, which is relevant to amphetamine, methamphetamine, cathinones, and morphine. Although chemogenetic receptors with higher potencies (EC₅₀<0.1 μM) require more extensive optimization, they have repeatedly been achieved.

### Example 4—Chemogenetic Modulation in Neurons in the Presence of Cocaine

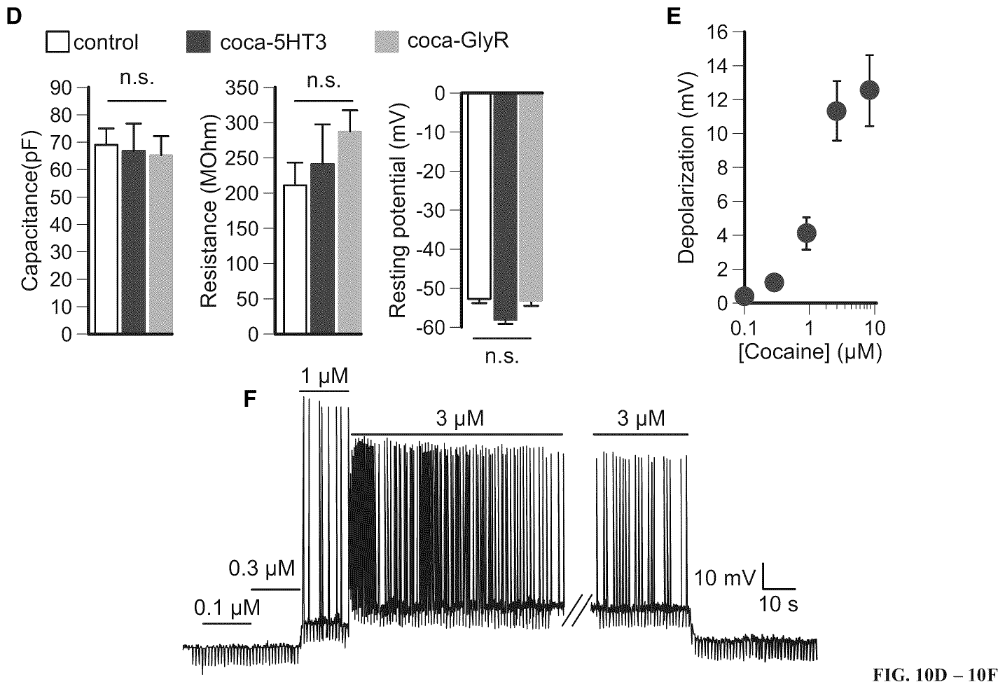

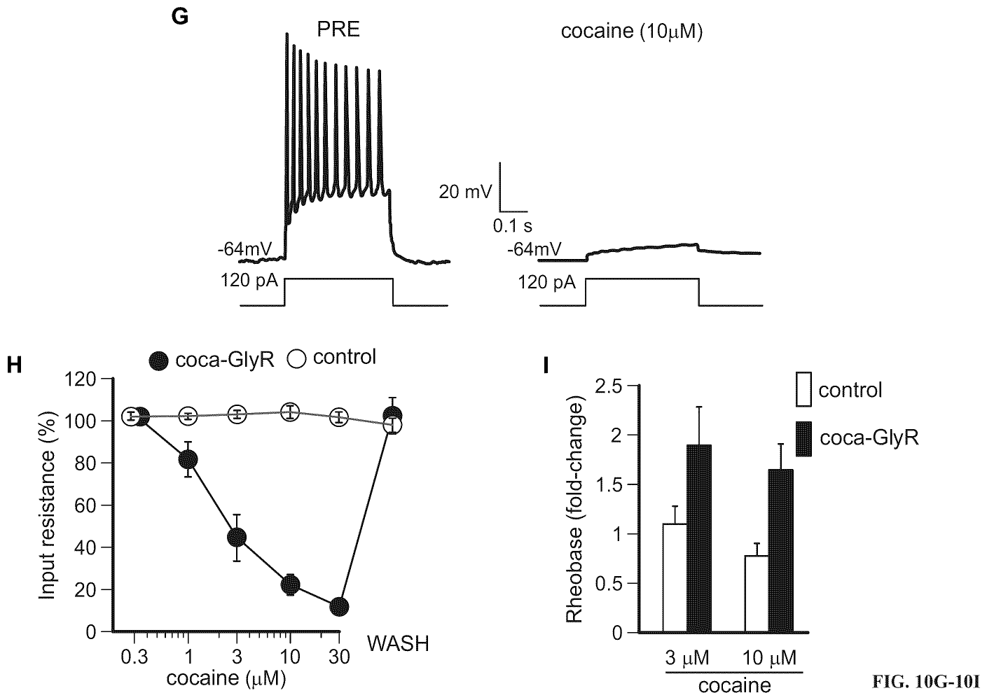

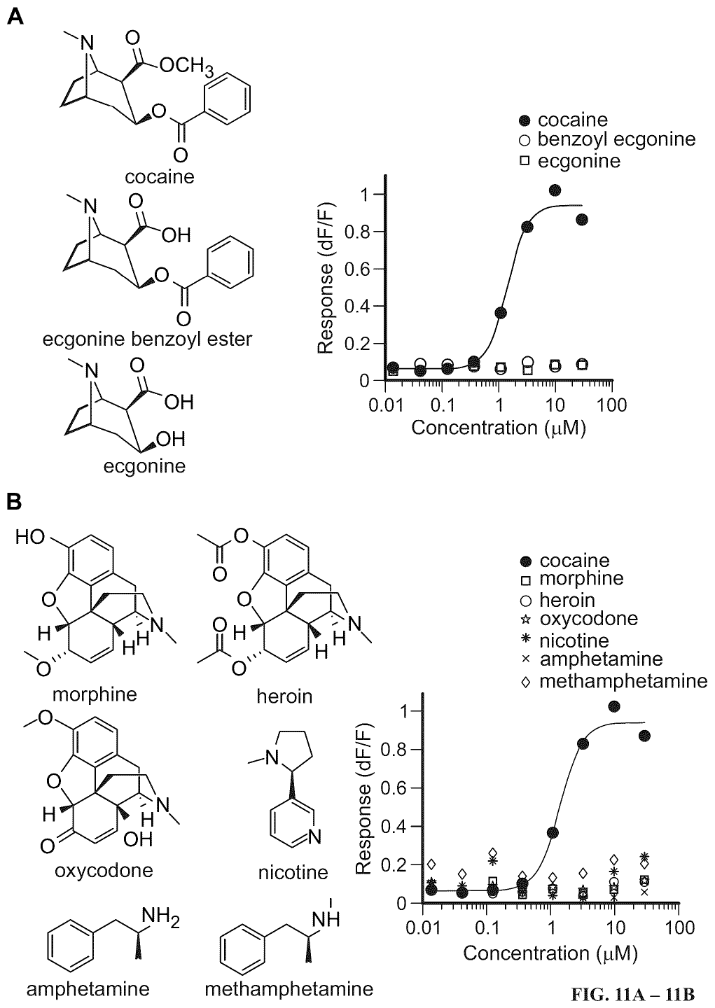

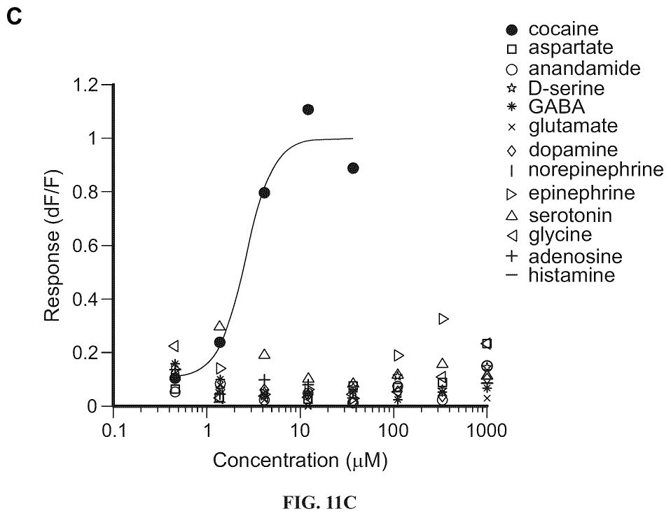

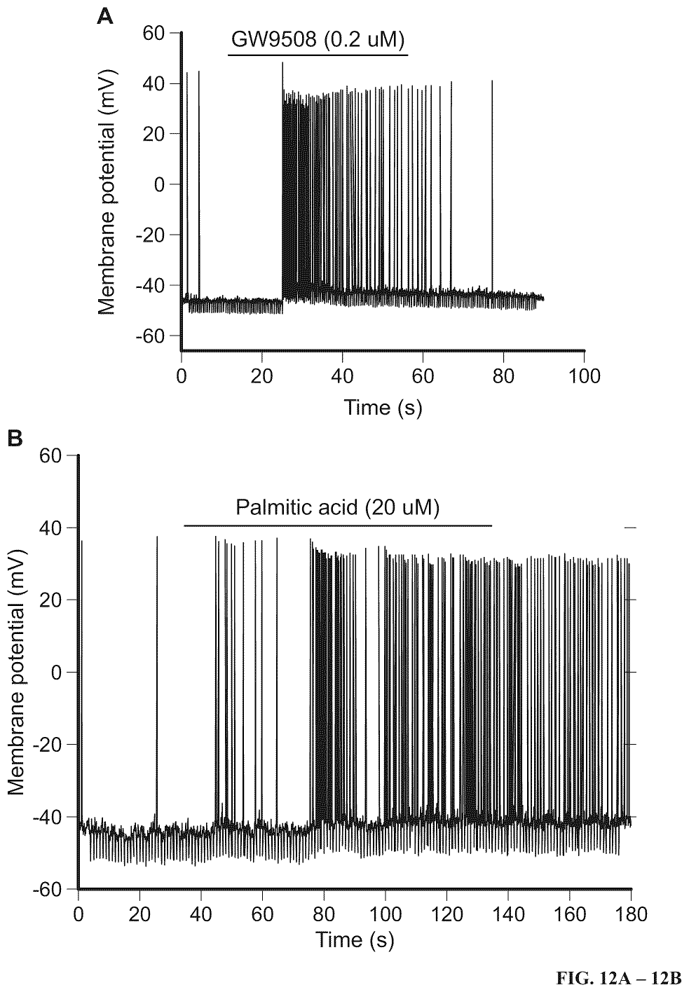

For optimization of a cocaine-sensitive chemogenetic receptor, the mutant chimeric channel, α7-5HT3 L141G, was selected from our screen and Type 2 and 3 mutations were used: the potency boosting mutation, Gly¹⁷⁵→Lys along with ACh-reducing mutations of two Tyr→Phe in the LBD. The resulting channel was called coca-5HT3, which showed good cocaine potency (EC50_{cocaine}: 1.5±0.3 μM) and affinity (Kᵢ: 0.033 μM) and right-shifted ACh potency (EC50_{Ach}: 216±35 μM) (FIG. 10A, 10B), along with high EC50_{choline}: >1 mM. These potencies are well above the brain concentrations of endogenous agonists. A similar set of mutations applied to α7-GlyR were used to produce coca-GlyR, a neuron silencing chloride channel (EC50_{cocaine}: 1.2±0.1 EC50_{Ach}: 64±12 EC50_{choline}: 245±12 μM, FIG. 10C). Potency for endogenous agonists is higher than measurements of transient ACh rises in the brain, which reach 1-2 μM and choline responsiveness was also low, requiring concentrations substantially higher than brain or circulating plasma levels (0.54 to 7.8 μM).

Neither of these channels show activation by the ecgonine or benzoyl ecgonine metabolites of cocaine. They are also selective for cocaine over other addictive drugs (EC₅₀>30 μM for nicotine, amphetamine, methamphetamine, morphine, heroin, oxycodone). These coca-IPD channels have been characterized by electrophysiology in HEK cells (FIG. 10B) and subsequently in neurons to establish that they do not perturb basal neuron properties (FIG. 10D).

The cocaine-responsive chemogenetic receptors are excellent neuromodulators. Coca-depolarizes hippocampal neurons (FIG. 10E) and leads to long-lasting action potential firing (FIG. 10F) at physiologically relevant cocaine concentrations. Coca-GlyR strongly suppressed evoked action potential firing in hippocampal neurons in the presence of cocaine (FIG. 10G) by substantially suppressing neuronal input resistance at a range of cocaine concentrations (FIG. 10H), thereby increasing the current amplitude required to elicit neuron firing (rheobase) (FIG. 10I).

The effect of drug-gated neuromodulators is examined by whole cell electrophysiology in neurons ex vivo to determine the influence of these channels on neuron polarization and firing. Cultured cortical neurons are transduced with AAV5-Syn::Ion_channel-IRES-mCherry or a corresponding mCherry control AAV. Two weeks post-transduction, resting membrane properties are measured and compared to the mCherry-expressing control neurons to assess resting membrane potential, input resistance, and membrane capacitance (as in FIG. 10). For neuron activation, the magnitude of depolarization is measured in response to increasing drug concentrations as well as excitability based on rheobase. For neuron inhibition, the change of input resistance is measured as is the rheobase from current injection to assess suppression of neuron excitability.

TABLE 2
Codon usage resulting in LBD amino acid substitutions

| WT amino | |
|---|---|
| acid residue | modified amino acid:modified nucleic acid codon |
| W77 | F:TTC |
| Q79 | A:GCA, E:GAG, F:TTC, G:GGC, M:ATG, N:AAC, |
| | Y:TAT |
| Y115 | F:TTC |
| L131 | G:GGA, N:AAC |
| Q139 | L:CTG, I:ATA |
| L141 | A:GCA, G:GGA, S:TCT, TCG, AGC, P:CCT, CCC |
| V154 | I:ATC |
| R155 | Y:TAC |
| W156 | N:AAC |
| H163 | T:ACC |
| S170 | F:TTC, G:GGA, H:CAT, L:CTC, N:AAC, T:ACC, |
| | V:GTA |
| S172 | A:GCA, V:GTA |
| G175 | K:AAA, S:TCA, V:GTA |
| Y210 | F:TTT |
| Y217 | A:GCC, F:TTC, H:CAT, K:AAA, R:AGA, S:TCA, |
| | V:GTA |

The above positions are relative to the following sequences and the relevant positions are shown with double underlining:

alpha 7-5HT3 (SEQ ID NO: 1), used to generate coca-5HT3:
MRCSPGGVWLALAASLLHVSLQGEFQRKLYKELVKNYNPLERPVANDSQPLTVYFSLSLLQIMDVDEK
NQVLTTNIWLQMSWTDHYLQWNVSEYPGVKTVRFPDGQIWKPDILLYNSADERFDATFHTNVLVNSSG
HCQYLPPGIFKSSCYIDVRWFPFDVQHCKLKFGSWSYGGWSLDLQMQEADISGYIPNGEWDLVGIPGK
RSERFYECCKEPYPDVTFTVIIRRRPLFYAVSLLLPSIFLMVVDIVGFCLPPDSGERVSFKITLLLGY
SVFLIIVSDTLPATIGTPLIGVYFVVCMALLVISLAETIFIVRLVHKQDLQRPVPDWLRHLVLDRIAW
ILCLGEQPMAHRPPATFQANKTDDCSGSDLLPAMGNHCSHVGGPQDLEKTPRGRGSPLPPPREASLAV
RGLLQELSSIRHFLEKRDEMREVARDWLRVGYVLDRLLFRIYLLAVLAYSITLVTLWSIWHYS
alpha 7-GlyR (SEQ ID NO: 2), used to generate coca-GlyR:
MRCSPGGVWLALAASLLHVSLQGEFQRKLYKELVKNYNPLERPVANDSQPLTVYFSLSLLQIMDVDEK
NQVLTTNIWLQMSWTDHYLQWNVSEYPGVKTVRFPDGQIWKPDILLYNSADERFDATFHTNVLVNSSG
HCQYLPPGIFKSSCYIDVRWFPFDVQHCKLKFGSWSYGGWSLDLQMQEADISGYIPNGEWDLVGIPGK
RSERFYECCKEPYPDVTFTVTMRRRMGYYLIQMYIPNLLIVILSWISEWINMDAAPARVGLGITTVLT
MTTQSSGSRASLPKVSYVKAIDIWMAVCLLFVESALLEYAAVNFVSRQHKELLRFRRKRRHHKEDEAG
EGRFNFSAYGMGPACLQAKDGISVKGANNSNTTNPPPAPSKSPEEMRKLFIQRAKKIDKISRIGFPMA
FLIFNMFYWIIYKIVRREDVHNQ

### Example 5—Chemogenetic Modulation in Neurons in the Presence of Fatty Acid

For engineered neuromodulation of neurons by fatty acids, a fatty acid transgene (Gpr40) was expressed in hippocampal neurons. Neuron firing was increased in response to a fatty acid, palmitate (20 μM), as well as a synthetic Gpr40 agonist, GW9508 (0.2 μM) (FIG. 12A, 12B). For testing, embryonic rat hippocampal neurons are transfected by electroporation with a plasmid expressing GPR40 and an mCherry fluorescent protein. Then cells are plated on poly-D-lysine coated 13 mm dia #1 glass coverslips in the wells of a 24 well plates at 50,000 cells/well. Each well receives 60 μL of cell suspension plus 60 μL of NbActiv4 medium (BrainBits) and are incubated at 37 C in 5% CO2 for 4 hours for initial cell attachment. NbActiv4 media (1 mL) is then added to each well and cultures kept for the duration of the experiment. Weekly medium changes are done by replacing 0.5 mL medium from each well with fresh NbActiv4. Fluorescent cells are recorded by patch clamp electrophysiology while palmitate or GW9508 are perfused over the cells intermittently in a saline extracellular solution.

Gpr40
(SEQ ID NO: 3)
MDLPPQLSFGLYVAAFALGFPLNVLAIRGATAHARLRLTPSLVYALNLGC
SDLLLTVSLPLKAVEALASGAWPLPASLCPVFAVAHFFPLYAGGGFLAAL
SAGRYLGAAFPLGYQAFRRPCYSWGVCAAIWALVLCHLGLVFGLEAPGGW
LDHSNTSLGINTPVNGSPVCLEAWDPASAGPARFSLSLLLFFLPLAITAF
CYVGCLRALARSGLTHRRKLRAAWVAGGALLTLLLCVGPYNASNVASFLY
PNLGGSWRKLGLITGAWSVVLNPLVTGYLGRGPGLKTVCAARTQGGKSQK

### Example 6—In Vivo Monitoring of Chemogenetic Neuromodulation

To measure chemogenetic neuromodulation, fiber photometry is used to monitor the activity of the neurons transduced by these chemogenetic receptors. an AAV construct, GCaMP7s:2a:coca-5HT3, has been developed and neuron activation and calcium elevation validated in response to cocaine. See, for example, FIG. 6C.

Neuromodulation is examined in vivo in the LHb and VTA DA neurons with fiber photometry. For LHb, an AAV with a pan-neuronal Synapsin promoter expressing both GCaMP7f and the chemogenetic ion channel (AAV5-Syn::Ion channel-IRES-GCaMP7f) is delivered. During the surgery, an optical fiber (Doric lenses) with a metal ferrule is implanted to minimize light loss. The LUX RZ10X processor and Synapse software fiber photometry system (Tucker-Davis Technologies) is used to measure calcium activity of neurons following i.p. injection of escalating doses of cocaine (saline, 5 mg/kg, 10 mg/kg, 20 mg/kg). These dose response curves are compared to control animals expressing only GCaMP7f. To test coca-GlyR in VTA^{DA} neurons, the Cre-dependent variant of the viral vector for use in Th::Cre rats is generated and viral transduction, fiber implantation, and dose responses are performed as described above.

For the same rats, to establish receptor occupancy for addictive drug doses at their cognate chemogenetic receptors, the clinical grade PET radioligand [¹⁸F]ASEM is used to detect coca-5HT3 (see above) and coca-GlyR. Receptor occupancy is measured following i.p. injection of escalating doses of cocaine (saline, 5 mg/kg, 10 mg/kg, 20 mg/kg). Rats and mice are scanned using a high-resolution small PET/CT scanner (Mediso USA).

### Example 7—In Vivo Pharmacology by Positron Emission Tomography (PET)

PET is a powerful method for non-invasive monitoring of receptor occupancy. It has previously been demonstrated that the well-validated α7 nAChR PET ligand [¹⁸F]-ASEM can bind chemogenetic ion channels. Analysis by PET in the presence of [¹⁸F]-ASEM followed by post hoc histology previously established the overlay of PET signal and chemogenetic receptor expression. It was found that [¹⁸F]-ASEM is also a ligand for coca-5HT3 (Kᵢ=9.3 nM). Thus, it is feasible to use this ligand to establish in vivo receptor occupancy by measuring displacement of [¹⁸F]-ASEM by cocaine to establish dose-occupancy relationships for chemogenetic targets in the brain.

### Example 8—IVSA and Negative-Feedback Chemogenetic Neuromodulation

It was tested whether coca-5HT3 expression in the LHb was sufficient to attenuate cocaine seeking behavior in rats using an intravenous (i.v.) cocaine self-administration (SA) procedure. Rats learned food pellet instrumental responding normally, and cocaine IVSA was similar to control rats. However, the dose response curve for IVSA showed significantly reduced responding for unit doses of cocaine on the ascending limb of the dose-response curve (where animals maintain high responding), indicating potential for treatment efficacy. For subsequent experiments, the shape of this curve likely will shift downward and upward for higher and lower potency chemogenetic receptors, respectively.

### Example 9—Whole Brain Activity Monitoring

Changes in whole brain metabolic activity in response to systemic cocaine injection (10 mg/kg, IP) was assessed in these rats expressing coca-5HT3 in LHb neurons using [¹⁸F]fluorodeoxyglucose (FDG) and PET.

### Example 10—Investigation of Neural Circuit Nodes that Modulate Drug Self-Administration

The IVSA model is used to investigate negative feedback control of drug-seeking by the LHb and the VTA^{DA} circuit nodes. Briefly, (a) lateral habenula neurons are targeted for cocaine-mediated activation in rats during cocaine self-administration using receptors with 3 different potency thresholds (EC₅₀: 0.3-0.5 μM, 1.5 μM, 7-10 μM); (b) VTA^{DA} neurons are targeted for cocaine-mediated inhibition during cocaine self-administration. Both groups will be compared to self-administration of food as a natural reward; (c) the LHb is targeted for negative-feedback neuronal activation and VTA dopamine neurons for inhibition with two additional chemogenetic systems identified from the screen using additional drugs from the class of nicotine, amphetamines, cathinones, or opioids; (d) For rats expressing excitatory chemogenetic receptors in the LHb as well as control rats, in vivo functional imaging with FDG is performed to compare brain network activation during cocaine administration in the presence and absence of chemogenetic neuromodulation.

### Example 11—Evaluation of Chemogenetic Receptors Engineered to Bind Amphetamine, Cocaine, Nicotine or Opioids

Multiple amino acid residues are mutated to identify chemogenetic receptors with increased potency for amphetamines and reduced potency for the endogenous agonist, ACh. The amphetamine derivatives MDMA (3,4-Methylenedioxy-methamphetamine, also known as ecstasy) and methamphetamine, do not show agonist activity at unmodified the chimeric receptors α7-5HT3. Both these drugs do show agonist activity at multiple chimeric receptors with mutated α7 nAChR ligand binding domains. Table 3 shows that mutations at Leu131 and Ser170 increase potency for MDMA and methamphetamine. An exemplary receptor, α7-L131G L141G G175S Y210F, has good MDMA potency (EC50 5 μM) and greatly reduced ACh potency (EC50 102 μM) at the chimeric receptor.

TABLE 3
EC50 (μM ± SEM) of amphetamines and acetylcholine (ACh) for chimeric
channels based on α7 nAChR ligand binding domain and 5HT3 or GlyR ion pore domain

| Chimeric channel | MDMA | Methamphetamine | ACh |
|---|---|---|---|
| α7-5HT3 | nd | nd | 10.7 ± 0.7 |
| α7-GlyR | nd | nd | 10.3 ± 1.6 |
| α7-5HT3 L131G S170T | >10 | nd | 4 |
| a7-5HT3 L131G L141G G175S Y210F | 5 | nd | 102 |
| a7-5HT3 L131N S172A G175S Y217F | >13 | nd | 18 |
| a7-5HT3 L131N S172V G175S Y217F | >12 | nd | 9 |
| a7-5HT3 L131N S172A G175K Y210F Y217F | 30 | 30 | 452 |
| a7-5HT3 L141G S170T | >10 | nd | 4 |
| a7-5HT3 S170F | >11 | nd | 4 |
| a7-5HT3 S170L | 11.3 | 11.3 | 1 |
| a7-5HT3 S170L Y217F | 7.9 | nd | 27 |
| a7-5HT3 S170T | 4.7 | 30 | 0.3 |
| a7-5HT3 S170T Y217F | >11 | nd | 9 |
| a7-5HT3 S170T G175K | 3.12 | 10.5 | <0.1 |
| a7-5HT3 Y115F S170T | >12 | nd | 9 |
| a7-5HT3 Q79G S170T G175K | 5.94 | 30 | <3 |
| a7-5HT3 Y115F S170T G175K | 8.9 | nd | 5 |
| a7-5HT3 S170T G175K Y210F | >10 | nd | 30 |
| a7-5HT3 S170T G175K Y217F | 6.2 | nd | <6 |
| a7-5HT3 S170V | 30 | nd | 3 |

nd, not detected
MDMA, 3,4-Methyl-enedioxy-methamphetamine

Multiple amino acid residues are mutated to identify chemogenetic receptors with increased potency for cocaine and reduced potency for the endogenous agonist, ACh. Cocaine does not show agonist activity at unmodified the chimeric receptors α7-5HT3 or α7-GlyR. Cocaine shows agonist activity at multiple chimeric receptors with mutated α7 nAChR ligand binding domains. Table 4 shows 101 receptors with cocaine agonist activity. Leu141→Gly is the most effective mutation for introducing cocaine agonist activity to the chimeric receptors. Cocaine agonism of mutated α7-5HT3 or α7-GlyR chimeric receptors is also associated with mutation of Leu141→Ala, Trp77→Phe, Tyr115→Phe and Glyl75→Ser, Gln79→Gly and Glyl75→Lys. Table 4 shows 35 receptors with cocaine EC50<5 μM and ACh EC50>100 μM (bolded). Each of these 35 receptors has the Leu141→Gly mutation. Potency of cocaine chemogenetic receptors can be increased (lower cocaine EC50) by addition of mutations at Gln79, Leu131, Gln139, Ser170, and Gly175. ACh potency is reduced (high ACh EC50) by addition of mutations at Tyr115, Val154, Arg155, Trp156, His163, Ser172, Tyr210, Tyr217. Table 4 also shows 10 exceptionally potent and selective cocaine activated channels that have cocaine EC50<1 μM and ACh EC50>100 μM, which, in addition to Leu141→Gly, have combinations of mutations including Val154→Ile, Arg155→Tyr, Trp156→Asn, His163→Thr, Ser172→Ala, Glyl75→Ser, Glyl75→Val, Ser172→Ala, Tyr210→Phe, Tyr217→Phe, Tyr217→Ser, Tyr217→Val Cocaine agonism of mutated α7-5HT3 or α7-GlyR chimeric receptors is also associated with mutation of Leu141→Ala, Leu141→Ser, Trp77→Phe, Tyr115→Phe and Glyl75→Ser, Gln79→Gly and Gly175→Lys.

TABLE 4
EC50 (μM ± SEM) of cocaine and acetylcholine (ACh) for chimeric
channels based on α7 nAChR ligand binding domain and 5HT3 or GlyR ion pore domain

| Chimeric channel | Cocaine | ACh |
|---|---|---|
| α7-5HT3 | nd | 10.7 ± 0.7 |
| α7-GlyR | nd | 10.3 ± 1.6 |
| α7-GlyR Y115F G175S | 10.4 | 3.9 |
| α7-GlyR Q79G G175K | 8.9 | 43.9 ± 24.1 |
| α7-5HT3 Q79E L141G | 6.7 | 37.2 |
| α7-5HT3 Q79F L141G | 3 | 19.2 |
| α7-5HT3 Q79M L141G | 2.5 | 15 |
| α7-5HT3 Q79Y L141G | 7.1 | 15.6 |
| α7-GlyR W77F | 3.4 | 7.8 |
| α7-5HT3 W77F L141A | 6.1 | 147 |
| α7-5HT3 W77F L141G | >12 | 38.1 |
| α7-GlyR W77F Q79G G175K | 4.4 ± 0.8 | 11.6 ± 2.9 |
| α7-GlyR W77F G175K | 0.57 | 1.5 |
| α7-GlyR W77F G175K Y210F | >10 | 81.3 |
| a7-5HT3 Q79G L141G | 8.2 | 42.6 |
| α7-GlyR Q79G L141G | >13 | 120 |
| α7-5HT3 L131G L141G | 4.9 ± 1.4 | 31.3 ± 9.0 |
| α7-5HT3 L131G L141G G175K Y115F | 11 | 130 |
| α7-5HT3 L131G L141G G175K Y210F | 5.9 | 222 |
| a7-5HT3 L131G L141G G175S Y115F | 3.3 | 30 |
| a7-5HT3 L131G L141G G175S Y210F | 1.23 | 102 |
| α7-5HT3 L131G L141G G175K Y217F | 1.1 ± 0.8 | 28 ± 5 |
| α7-5HT3 L131G L141G G175K Y217S | 10 | 150 |
| α7-5HT3 L131G L141G G175S Y217S | 4.2 | 108 |
| α7-5HT3 L131G L141G G175V Y217S | 15 | 6 |
| α7-5HT3 L131G L141G G175V Y217V | 10 | 1 |
| α7-5HT3 L131G L141G G175S Y217V | 3.9 | 300 |
| α7-5HT3 L131G L141G G175K Y217V | 20 | 26 |
| α7-5HT3 L131G L141G G175S Y217V W156N H163T | 1.2 | 213 |
| α7-5HT3 L131G L141G G175S Y217V H163T | 0.48 | 342 |
| a7-5HT3 L131G L141G G175K Y217F V154I R155Y W156N | 1.4 | 33 |
| α7-5HT3 L131G S170T | 0.3 | 4 |
| a7-5HT3 L131N S170T G175K Y217F | 8.3 | 6 |
| a7-5HT3 L131N S172A G175K Y210F Y217F | 30 | 452 |
| α7-GlyR L131N | >10 | 10.2 |
| α7-5HT3 L141A | 3.5 | 3.3 |
| α7-GlyR L141A | 4.5 ± 1.5 | 26.2 ± 5.8 |
| α7-5HT3 L141G | 2.4 ± 0.01 | 31.2 ± 0.8 |
| α7-GlyR L141G | 4.4 ± 0.9 | 50.0 ± 6.4 |
| α7-GlyR Q139I L141G | 2.6 ± 0.4 | 50.6 ± 2.6 |
| α7-GlyR Q139L L141G | 0.9 ± 0.2 | 26.0 ± 2.9 |
| α7-5HT3 L141G G175K | 0.32 ± 0.08 | 1.9 ± 0.1 |
| α7-GlyR L141G G175K | 1.0 ± 0.1 | 11.0 ± 0.7 |
| α7-5HT3 L141G G175K Y115F | 6.1 ± 0.7 | 82.7 ± 14.0 |
| α7-GlyR L141G G175K Y115F | 5.9 | 157 |
| α7-5HT3 L141G G175K Y210F | 2.3 ± 0.7 | 158 ± 35 |
| α7-GlyR L141G G175K Y210F | 4.8 ± 0.6 | 496 ± 154 |
| α7-GlyR L141G G175K Y210F Y217F | 3.0 ± 0.1 | 380 ± 145 |
| α7-5HT3 L141G G175K Y210F Y217F | 1.6 ± 0.3 | 236 ± 30 |
| α7-5HT3 L141G G175K Y217A | 6.7 | 1100 |
| α7-5HT3 L141G G175K Y217F | 0.7 ± 0.1 | 17.8 ± 3.2 |
| α7-GlyR L141G G175K Y217F | 1.3 ± 0.1 | 65.4 ± 10.1 |
| α7-GlyR L141G G175K Y217S | 4.5 | nd |
| α7-5HT3 L141G G175S Y217S | 1.75 | 300 |
| α7-GlyR L141G G175S Y217S | 9.8 | 3000 |
| α7-5HT3 L141G G175V Y217S | 4.5 | 200 |
| α7-GlyR L141G G175V Y217S | 12.5 | 13 |
| α7-5HT3 L141G G175S Y217V | 0.72 | 110 |
| α7-GlyR L141G G175S Y217V | 1.35 | 232 |
| α7-5HT3 L141G G175V Y217V | 10 | 37 |
| α7-GlyR L141G G175V Y217V | 9.5 | 1000 |
| α7-GlyR L141G G175K Y217V | 3.8 | nd |
| α7-5HT3 L141G G175S Y210F Y217S | 3.66 | 113 |
| α7-5HT3 L141G G175S Y210F Y217V | 2.8 | 1000 |
| α7-5HT3 L141G G175V Y210F Y217S | 10 | 28 |
| α7-5HT3 L141G G175V Y210F Y217V | 6.2 | 26 |
| α7-5HT3 L141G G175S Y210F Y217F | 0.15 | 120 |
| α7-5HT3 L141G G175V Y210F Y217F | 0.165 | 80 |
| a7-5HT3 L141G S170T | 0.3 | 4 |
| a7-5HT3 L141G S170T G175K Y210F | 0.57 | 53 |
| a7-5HT3 L141G S170T G175K Y210F Y217F | 1.5 | 228 |
| a7-5HT3 L141G S170T S172V G175K Y210F | 3.9 | 300 |
| a7-5HT3 L141G S170T S172A G175K Y210F | 2.49 | 228 |
| a7-5HT3 L141G S170T S172A G175K Y210F Y217F | 3.27 | 807 |
| a7-5HT3 L141G S170T G175K Y217F | 0.24 | 8 |
| α7-GlyR L141G S172A G175S | 0.42 | 12 |
| α7-GlyR L141G S172A G175K Y217F | 2.4 ± 0.8 | 124 |
| α7-GlyR L141G S172A G175S Y210F | 1.89 | 579 |
| α7-GlyR L141G S172A G175S Y217F | 0.6 ± 0.3 | 61.5 ± 1.5 |
| α7-GlyR L141G S172A G175S Y210F Y217F | 1.38 | 390 |
| α7-5HT3 L141G S172A G175S Y217S | 4.7 | 107 |
| α7-5HT3 L141G S172A G175S Y210F Y217F | 0.54 | 139 |
| α7-5HT3 L141G G175K Y210F Y217F W156N H163T | 2.61 | 261 |
| α7-5HT3 L141G G175K Y210F Y217F H163T | 1.29 | 290 |
| a7-5HT3 L141G G175S Y210F Y217F V1541 R155Y W156N | 0.33 | 105 |
| α7-5HT3 L141G G175S Y210F Y217F W156N H163T | 0.8 | 127 |
| α7-5HT3 L141G G175S Y210F Y217F H163T | 0.36 | 92 |
| a7-5HT3 L141G W156N H163T G175S Y217V | 1.5 ± 0.3 | 113 |
| a7-5HT3 L141G H163T G175S Y217V | 0.7 ± 0.2 | 360 ± 18 |
| α7-5HT3 L141G G175S Y217V V154I R155Y W156N | 0.69 | 422 |
| α7-5HT3 L141G G175V Y210F Y217F H163T | 0.51 | 177 |
| α7-5HT3 L141G G175V Y210F Y217F V1541 R155Y W156N) | 0.4 | 157 |
| α7-5HT3 L141G G175V Y210F Y217F W156N H163T | 0.87 | 184 |
| α7-5HT3 L141S G175K | 7.3 | 8.4 |
| a7-5HT3 Q139L L141G | 0.36 | 4.2 |
| a7-5HT3 Q1391 L141G | 2.8 | 13.8 |
| a7-5HT3 S170L | 5.5 | 1 |
| a7-5HT3 S170L Y210F | 1.41 | 33 |
| a7-5HT3 S170L Y217F | 2.8 | 27 |
| a7-5HT3 S170P | 4.17 | 10 |
| a7-5HT3 S170T Y210F | nd | 54 ± 5.5 |
| a7-5HT3 S170T Y217F | 5.22 | 9 |
| a7-5HT3 S170V Y115F | >8 | 27 |
| a7-5HT3 S170V Y217F | 6.36 | 39 |

nd, not detected

Multiple amino acid residues are mutated to identify chemogenetic receptors with increased potency for nicotine. Nicotine shows activity for unmodified α7-5HT3 (EC50: 6.7 μM) and α7-GlyR (EC50: 6.8 μM) chimeric receptors. Table 5 shows that nicotine activity for chimeric receptors is reduced by mutations at Leu131, Ser170, and Gly175. ACh potency is reduced by mutations at Tyr210 and Tyr217.

TABLE 5
EC50 (μM ± SEM) of nicotine and acetylcholine
(ACh) for chimeric channels based on α7 nAChR
ligand binding domain and 5HT3 or GlyR ion pore domain

| Chimeric channel | Nicotine | ACh |
|---|---|---|
| α7-5HT3 | 6.7 ± 2.8 | 10.7 ± 0.7 |
| α7-GlyR | 6.8 ± 2.2 | 10.3 ± 1.6 |
| a7-5HT3 Q79A | 1.2 | 13 |
| a7-5HT3 Q79G | 2 | 44 |
| α7-GlyR L131N | 4.2 | 10.2 ± 4.2 |
| α7-5HT3 L131G L141G | 7.4 | 31.3 ± 9.0 |
| α7-5HT3 L131G S170T | 0.9 | 4 |
| α7-5HT3 L131N G175K | 0.1 | 8 |
| α7-5HT3 L131N G175K Y217F | 0.7 | 440 |
| a7-5HT3 L131N S170T G175K Y217F | 0.21 | 6 |
| a7-5HT3 L131N S172A G175K Y217F | 1.2 | 27 |
| a7-5HT3 L131N S170T S172A G175K Y217F | 2.64 | 78 |
| a7-5HT3 L131N S172A G175S Y217F | 0.87 | 18 |
| a7-5HT3 L131N S172V G175S Y217F | 0.54 | 9 |
| a7-5HT3 L131N S172A G175K Y210F Y217F | 12.7 | 452 |
| α7-GlyR Q139L L141G | 3.8 ± 2.4 | 26.0 ± 2.9 |
| α7-5HT3 L141G G175K | 1.11 | 1.9 ± 0.1 |
| a7-5HT3 L141G S170T | 0.9 | 4 |
| a7-5HT3 L141G S170T G175K Y210F | 3.95 | 53 |
| a7-5HT3 L141G S170T S172A G175K Y210F | 6.9 | 228 |
| a7-5HT3 L141G S170T G175K Y217F | 2.22 | 8 |
| α7-5HT3 L141G G175S Y210F Y217F | 4.2 | 120 |
| α7-GlyR L141G S172A G175S | 4.3 | 12 |
| a7-5HT3 S170F | 1.1 | 4 |
| a7-5HT3 S170G | 1 | 4 |
| a7-5HT3 S170H | 2.4 | 8 |
| a7-5HT3 S170L | 0.3 | 1 |
| a7-5HT3 S170L Y115F | 3.75 | 45 |
| a7-5HT3 S170L Y210F | >10.2 | 33 |
| a7-5HT3 S170L Y217F | 2.28 | 27 |
| a7-5HT3 S170P | 4 | 10 |
| a7-5HT3 S170T | 0.09 | 0.3 |
| a7-5HT3 S170T Y210F | 3.5 ± 0.5 | 54.0 ± 5.5 |
| a7-5HT3 S170T Y217F | 1.35 | 9 |
| a7-5HT3 S170T G175K | <0.1 | <0.1 |
| a7-5HT3 Y115F S170T | 1.08 | 9 |
| a7-5HT3 Q79G S170T G175K | <0.1 | <3 |
| a7-5HT3 Y115F S170T G175K | 0.21 | 5 |
| a7-5HT3 S170T G175K Y210F | 1.1 | 30 |
| a7-5HT3 S170T G175K Y217F | 0.45 | <6 |
| a7-5HT3 S170V | 0.84 | 3 |
| a7-5HT3 S170V Y115F | 3.87 | 27 |
| a7-5HT3 S170V Y210F | 8.1 | 114 |
| a7-5HT3 S172A | 3.37 | 30 |
| a7-5HT3 S172I | 5.4 | 48.8 ± 16.8 |
| a7-5HT3 S172V | 4.6 | 31.3 ± 12.9 |

Multiple amino acid residues are mutated to identify chemogenetic receptors with increased potency for opioids. Opioids do not show activity for unmodified α7-5HT3 and α7-GlyR chimeric receptors. Table 6 shows examples of mutated chimeric channels with agonist activity from fentanyl, oxycodone, morphine, or heroin. Increased opioid potency is associated with mutation of Trp77, Leu131, Leu141, Ser170, Gly175. ACh potency is reduced by addition of mutations at Tyr115, Val154, Arg155, Trp156, His163, Ser172, Tyr210, Tyr217. Exemplary chimeric potency for fentanyl (EC50<1 μM) and selectivity over ACh (EC50>100 μM) was shown for 5 chimeric channels (bolded below).

TABLE 6
EC50 (μM ± SEM) of opioids and acetylcholine (ACh) for chimeric
channels based on α7 nAChR ligand binding domain and 5HT3 or GlyR ion pore domain

| Chimeric channel | fentanyl | oxycodone | morphine | heroin | ACh |
|---|---|---|---|---|---|
| α7-5HT3 | nd | nd | nd | nd | 10.7 ± 0.7 |
| α7-GlyR | nd | nd | nd | nd | 10.3 ± 1.6 |
| α7-5HT3 W77F L141A | 9.7 ± 2.1 | nd | nd | nd | 147 |
| α7-5HT3 W77F L141G | >10 | nd | nd | nd | 38.1 |
| α7-5HT3 Q79M L141G | >13 | nd | nd | nd | 15 |
| α7-5HT3 Q79Y L141G | >12 | nd | nd | nd | 15.6 |
| a7-5HT3 Q79G L141G | >12 | nd | nd | nd | 42.6 |
| α7-GlyR L131G Y217F | >12 | nd | nd | nd | 57.0 ± 20.7 |
| α7-GlyR L131V | >10 | nd | nd | nd | 10.8 |
| α7-5HT3 L131G L141G | 1.1 ± 0.1 | nd | nd | nd | 31.3 ± 9.0 |
| α7-5HT3 L131G L141G G175K | 0.17 | nd | nd | nd | 130 |
| Y115F | | | | | |
| α7-5HT3 L131G L141G G175K | 0.23 | nd | nd | nd | 222 |
| Y210F | | | | | |
| α7-5HT3 L131G L141G G175K | 0.12 ± 0.02 | nd | nd | nd | 28 |
| Y217F | | | | | |
| α7-5HT3 L131G L141G G175V | 10 | nd | nd | nd | 1 |
| Y217V | | | | | |
| α7-5HT3 L131G L141G G175S | 7.5 | nd | nd | nd | 300 |
| Y217V | | | | | |
| α7-5HT3 L131G L141G G175K | 10 | nd | nd | nd | 26 |
| Y217V | | | | | |
| α7-5HT3 L131G L141G G175K | 10 | nd | nd | nd | 150 |
| Y217S | | | | | |
| α7-5HT3 L131G L141G G175S | 8.2 | nd | nd | nd | 108 |
| Y217S | | | | | |
| α7-5HT3 L131G L141G G175V | 10 | nd | nd | nd | 6 |
| Y217S | | | | | |
| α7-5HT3 L131G S170T | 3 | 5.5 | nd | nd | 4 |
| a7-5HT3 L131G L141G S172A | 0.36 | nd | nd | nd | 273 |
| G175K | | | | | |
| a7-5HT3 L131G L141G S172A | 1.44 | nd | nd | nd | 440 |
| G175K V154I R155Y W156N | | | | | |
| α7-5HT3 L131G L141G S172V | 0.24 | nd | nd | nd | 291 |
| G175K Y115F | | | | | |
| a7-5HT3 L131G L141G G175K | 0.36 | nd | nd | nd | 33 |
| Y217F V154I R155Y W156N | | | | | |
| a7-5HT3 L131G L141G G175S | 0.06 | nd | nd | nd | 30 |
| Y115F | | | | | |
| a7-5HT3 L131G L141G G175S | 0.15 | nd | nd | nd | 102 |
| Y210F | | | | | |
| a7-5HT3 L131N S172A G175K | 9.45 | nd | nd | nd | 27 |
| Y217F | | | | | |
| a7-5HT3 L131N S172V G175S | >12 | nd | nd | nd | 9 |
| Y217F | | | | | |
| α7-GlyR Q139L L141G | 10.6 ± 4.3 | nd | nd | nd | 26.0 ± 2.9 |
| α7-5HT3 L141G | >13 | nd | nd | nd | 31.2 ± 0.8 |
| α7-GlyR L141G | >10 | nd | nd | nd | 50.0 ± 6.4 |
| α7-5HT3 L141G G175K | 1.6 ± 0.6 | nd | nd | nd | 1.9 ± 0.1 |
| α7-GlyR L141G G175K | 4.6 ± 0.7 | nd | nd | nd | 11.0 ± 0.7 |
| α7-5HT3 L141G G175K Y115F | 4.3 ± 0.9 | nd | nd | nd | 82.7 ± 14.0 |
| α7-GlyR L141G G175K Y115F | 5 | nd | nd | nd | 157 |
| α7-5HT3 L141G G175K Y210F | 6.7 ± 0.8 | nd | nd | nd | 158 ± 35 |
| α7-GlyR L141G G175K Y210F | >13 | nd | nd | nd | 496 ± 154 |
| α7-5HT3 L141G G175K Y217F | 4.9 ± 1.0 | nd | nd | nd | 17.8 ± 3.2 |
| α7-GlyR L141G G175K Y217F | >13 | nd | nd | nd | 65.4 ± 10.1 |
| α7-GlyR L141G S172A G175K | 12 | nd | nd | nd | 124 |
| Y217F | | | | | |
| α7-GlyR L141G S172A G175S | >12 | nd | nd | nd | 61.5 ± 1.5 |
| Y217F | | | | | |
| a7-5HT3 L141G S170T | 3 | nd | nd | nd | 4 |
| a7-5HT3 L141G S170T G175K | 4.1 | >11 | nd | nd | 53 |
| Y210F | | | | | |
| a7-5HT3 L141G S170T G175K | 5.04 | nd | nd | nd | 228 |
| Y210F Y217F | | | | | |
| a7-5HT3 L141G S170T S172V | >9 | nd | nd | nd | 300 |
| G175K Y210F | | | | | |
| a7-5HT3 L141G S170T S172A | 5.73 | nd | nd | 8.9 | 228 |
| G175K Y210F | | | | | |
| a7-5HT3 L141G S170T S172A | >10 | nd | nd | nd | 807 |
| G175K Y210F Y217F | | | | | |
| a7-5HT3 L141G S170T G175K | 5.1 | nd | nd | nd | 8 |
| Y217F | | | | | |
| α7-5HT3 L141G G175K Y210F | >10 | nd | nd | nd | 236 ± 30 |
| Y217F | | | | | |
| α7-GlyR L141G G175K Y210F | >13 | nd | nd | nd | 380 ± 146 |
| Y217F | | | | | |
| α7-5HT3 L141G G175V Y210F | 10 | nd | nd | nd | 28 |
| Y217S | | | | | |
| α7-5HT3 L141G G175V Y210F | 10 | nd | nd | nd | 26 |
| Y217V | | | | | |
| α7-5HT3 L141G G175S Y210F | 4.8 | nd | nd | nd | 120 |
| Y217F | | | | | |
| α7-5HT3 L141G G175V Y210F | 3 | nd | nd | nd | 80 |
| Y217F | | | | | |
| α7-5HT3 L141G G175K Y210F | 12.6 | nd | nd | nd | 290 |
| Y217F H163T | | | | | |
| a7-5HT3 L141G G175S Y210F | 13.1 | nd | nd | nd | 105 |
| Y217F V154I R155Y W156N | | | | | |
| α7-5HT3 L141G G175S Y210F | 11.3 | nd | nd | nd | 92 |
| Y217F H163T | | | | | |
| α7-5HT3 L141G G175V Y210F | 5.25 | nd | nd | nd | 177 |
| Y217F H163T | | | | | |
| α7-5HT3 L141G G175V Y210F | 12.1 | nd | nd | nd | 184 |
| Y217F W156N H163T | | | | | |
| a7-5HT3 Q139L L141A | >15 | 12.8 | nd | nd | 8.1 |
| a7-5HT3 S170E | >12 | nd | nd | nd | 48 |
| a7-5HT3 S170F | >12 | nd | nd | nd | 4 |
| a7-5HT3 S170L | nd | 11 | nd | nd | 1 |
| a7-5HT3 S170L Y115F | >14 | nd | nd | nd | 45 |
| a7-5HT3 S170N | 12.5 | nd | 12.5 | nd | 11 |
| a7-5HT3 S170T | 30 | 11.1 | nd | nd | 0.3 |
| a7-5HT3 S170V | 30 | 6.4 | nd | nd | 3 |
| a7-5HT3 S170V Y115F | >10 | 8.3 | nd | nd | 27 |

nd, not detected

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. An engineered human chemogenetic cell-surface receptor comprising:
a ligand binding domain (LBD) and an activation domain, wherein the LBD has been engineered to bind a ligand associated with an ingested substance whose ingestion results in reinforcing behavior.

2. The receptor of claim 1, wherein the ingested substance is a controlled substance or a nutrient substance.

3. The receptor of claim 2, wherein the controlled substance is an addictive drug.

4. The receptor of claim 2, wherein the nutrient substance is a sugar, caffeine, or a fatty acid.

5. The receptor of claim 2, wherein the controlled substance is selected from the group consisting of cocaine or cocaine metabolites, methylphenidate (Ritalin), amphetamine, cathinone, and opioid.

6. The receptor of claim 5, wherein the amphetamine is selected from amphetamine, MDMA, and methamphetamine.

7. The receptor of claim 5, wherein the cathinone is selected from bupropion, MDPV, mephedrone, and methylone.

8. The receptor of claim 5, wherein the opioid is selected from morphine, oxycodone, dihydrocodeine, heroin, methadone, and fentanyl.

9. The receptor of claim 1, wherein the cell-surface receptor is selected from a ligand gated ion channel (LGIC) or a G-protein coupled receptor (GPCR).

10. The receptor of claim 1, wherein the LBD is a mutated alpha-7-5HT3 LBD.

11. The receptor of claim 1, wherein the LBD is a mutated alpha 7-GlyR LBD.

12. A cell comprising the engineered human chemogenetic cell-surface receptor of claim 1.

13. The cell of claim 12, wherein the cell is a neuron.

14. The cell of claim 12, wherein the cell is in culture.

15. The cell of claim 12, wherein the cell is in vivo.

16. A method of treating a disorder associated with the use of an ingested substance, comprising:
delivering the engineered human chemogenetic cell-surface receptor of claim 1 to an individual,
wherein, in the presence of the ingested substance, the engineered human chemogenetic cell-surface receptor reduces a reward response for the ingested substance or increases an aversion response for the ingested substance.

17. The method of claim 16, wherein the ingested substance is a controlled substance or a nutrient substance.

18. The method of claim 17, wherein the controlled substance is an addictive drug.

19. The method of claim 17, wherein the nutrient substance is sugar, caffeine, or a fatty acid.

20. The method of claim 16, wherein the engineered human chemogenetic cell-surface receptor is delivered in the form of a nucleic acid encoding the engineered human chemogenetic cell-surface receptor.

21. The method of claim 17, wherein the controlled substance is selected from the group consisting of cocaine or cocaine metabolites, methylphenidate (Ritalin), amphetamine, cathinone, and opioid.

22. The method of claim 21, wherein the amphetamine is selected from amphetamine, MDMA, and methamphetamine.

23. The method of claim 21, wherein the cathinone is selected from bupropion, MDPV, mephedrone, and methylone.

24. The method of claim 21, wherein the opioid is selected from morphine, oxycodone, dihydrocodeine, heroin, methadone, and fentanyl.
